(19) United States
(12) Patent Application Publication
(10) Pub. No.: US 2010/0016379 A1
(43) Pub. Date: Jan. 21, 2010

(54) Terephthalamate Compounds and Compositions, and Their Use as HIV Integrase Inhibitors

(75) Inventors: Yun He, Shanghai (CN); Zhiwei Wang, Carlsbad, CA (US); Baogen Wu, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton, BM (BM)

(21) Appl. No.: 12/300,171

(22) PCT Filed: Apr. 18, 2007
(86) PCT No.: PCT/US07/09540

§ 371 Date: Feb. 18, 2009

Related U.S. Application Data
(60) Provisional application No. 60/747,262, filed on May 15, 2006.

(51) Int. Cl. A61K 31/195; C07C 229/38; C07D 213/56; A61K 31/44

(52) U.S. Cl. 514/357; 560/42; 546/337; 514/563

(57) ABSTRACT

Described herein are compounds having a terephthalamate structural feature. Also described herein, are methods of making such compounds, methods of using such compounds to modulate the activity of HIV integase, and pharmaceutical compositions and medicaments comprising such compounds. Also described herein are methods of using such compounds, pharmaceutical compositions and medicaments to treat and/or prevent and/or inhibit and/or ameliorate the pathology and/or symptomology of AIDS or infection with HIV.

### CROSS-REFERENCE

This application claims the benefit of U.S. provisional application Ser. No. 60/747,262 filed May 15, 2006, which is incorporated by reference in its entirety.

### FIELD OF THE INVENTION

Compounds, methods of making such compounds, pharmaceutical compositions and medicaments comprising such compounds, and methods of using such compounds to treat or prevent diseases or conditions associated with HIV integrase activity are described.

### BACKGROUND OF THE INVENTION

Human immunodeficiency virus (HIV), a retrovirus, is the etiological agent of acquired immune deficiency syndrome (AIDS). Several viral enzymes are essential for HIV replication including, but not limited to, reverse transcriptase, protease, and integrase. In particular, HIV integrase mediates the insertion of proviral DNA into the host cell genome. Inhibition of the strand transfer reactions catalyzed by recombinant integrase in HIV infected cells, results in integrase inhibition and impedes subsequent HIV replication. Viral enzyme inhibitors inhibiting HIV replication are useful agents in the treatment of AIDS and similar diseases, (for example, reverse transcriptase inhibitors such as Zidovudine (AZT) and Efavirenz; protease inhibitors such as Indinavir (IDV) and Nelfinavir).

### SUMMARY OF THE INVENTION

Described are compounds, methods of making such compounds, pharmaceutical compositions and medicaments comprising such compounds, and methods of using such compounds to treat or prevent diseases or conditions associated with HIV integrase activity.

In one aspect are compounds having the structure of Formula (I):

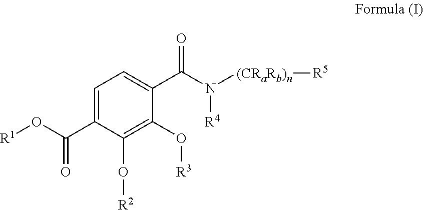

wherein

R¹ is H, alkyl or substituted alkyl;

R² is H, alkyl, substituted alkyl, —C(O)-alkyl or —C(O)-substituted alkyl;

R³ is H, alkyl, substituted alkyl, —C(O)-alkyl or —C(O)-substituted alkyl;

R⁴ is H, alkyl or substituted alkyl;

or —O—R³—R⁴—N— together form an optionally substituted, 6 or 7 membered ring;

Rₐ is H, halogen, C₁-C₆ alkyl or C₁-C₆ substituted alkyl;

R_{b} is H, halogen, C₁-C₆ alkyl or C₁-C₆ substituted alkyl;

R⁵ is optionally substituted C₃-C₅ cycloalkyl, optionally substituted lower heterocycloalkyl, optionally substituted aryl or optionally substituted heteroaryl;

where each substituent is independently selected from the group consisting of halogen, —CN,

—NO₂, —N₃, ═O, ═S, ═NH, —SO₂, nitroalkyl, amino, dialkylamino, diarylamino, diarylalkylamino, cyanato, isocyanato, thiocyanato, isothiocyanato, guanidinyl, O-carbamyl, N-carbamyl, thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, -L¹-H, -L¹-alkyl, -L¹-substituted alkyl, -L¹-heteroalkyl, -L¹-haloalkyl, -L¹-perhaloalkyl,
-L¹-alkenyl, -L¹-substituted alkenyl, -L¹-heteroalkenyl, -L¹-haloalkenyl, -L¹-perhaloalkenyl, -L¹-alkynyl, -L¹-substituted alkynyl, -L¹-heteroalkynyl, -L¹-haloalkynyl, -L¹-perhaloalkynyl, -L¹-cycloalkyl, -L¹-substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-unsubstituted aryl,
-L¹-heteroaryl, and -L¹-substituted heteroaryl;

where -L¹- is a bond, -alkylene-, -heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—,

### —C(S)NH—, —NHC(S)—, —S(O)—, —S(O)₂— or —S(O)NH—;

n is 0, 1 or 2; and a pharmaceutically acceptable salt, pharmaceutically acceptable N-oxide, pharmaceutically active metabolite, pharmaceutically acceptable prodrug, pharmaceutically acceptable solvate thereof.

In a further or alternative embodiment, R¹ is not H. In a further or alternative embodiment, R² and R³ are not methyl. In a further or alternative embodiment, R¹ is not H; and R² and R³ are not methyl. In a further or alternative embodiment, R⁴ is not H. In a further or alternative embodiment, R¹ is not H; R² and R³ are not methyl; and R⁴ is not H. In a further or alternative embodiment, R⁵ is not unsubstituted phenyl. In a further or alternative embodiment, R¹ is not H; R² and R³ are not methyl; R⁴ is not H; and R⁵ is not unsubstituted phenyl. In a further or alternative embodiment, compounds of Formula (I) are with a proviso that when R¹ is H; and R² and R³ are methyl, then R⁴ is not H; and R⁵ is not unsubstituted phenyl.

In a further or alternative embodiment, R¹ is alkyl. In a further or alternative embodiment, R¹ is H, methyl, ethyl, propyl, iso-propyl, butyl, iso-butyl, or tert-butyl. In a further or alternative embodiment, R¹ is H or methyl. In a further or alternative embodiment, R¹ is methyl. In a further or alternative embodiment, R¹ is H. In a further or alternative embodiment, R² is H. In a further or alternative embodiment, R³ is H. In a further or alternative embodiment, R² and R³ are H. In a further or alternative embodiment, R⁴ is H, methyl, ethyl, propyl, iso-propyl, butyl, iso-butyl, or tert-butyl. In a further or alternative embodiment, R⁴ is H or methyl. In a further or alternative embodiment, R⁴ is H. In a further or alternative embodiment, R⁴ is methyl. In a further or alternative embodiment, n is 0. In a further or alternative embodiment, n is 1.

In a further or alternative embodiment, R⁵ is optionally substituted aryl or optionally substituted heteroaryl. In a further or alternative embodiment, R⁵ is substituted aryl or optionally substituted heteroaryl. In a further or alternative embodiment, R⁵ is substituted phenyl or optionally substituted pyridyl. In a further or alternative embodiment, R⁵ is an unsubstituted phenyl or an unsubstituted pyridyl. In a further or alternative embodiment, R⁵ is substituted with at least one group selected from C₁-C₆ alkoxy, C₁-C₆ alkyl, C₁-C₆ haloalkyl, OH, NO₂, or NH₂. In a further or alternative embodiment, R⁵ is selected from the group consisting of:

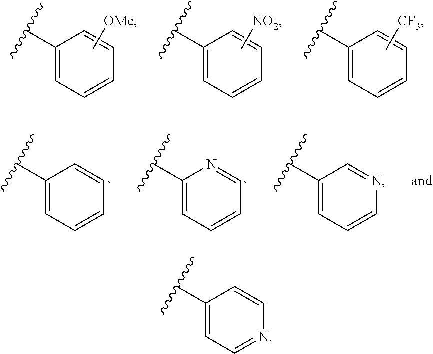

In a further or alternative embodiment, R¹ is alkyl; R²═R³═R⁴═H; R⁵ is substituted phenyl or substituted pyridyl; and n is 0 or 1. In a further or alternative embodiment, R¹ is alkyl; R²═R³═R⁴═H; R⁵ is unsubstituted phenyl or unsubstituted pyridyl; and n is 0 or 1. In a further or alternative embodiment, —O—R³—R⁴—N— together form an optionally substituted, 6 or 7 membered ring.

In another aspect are compounds having the structure of Formula (II):

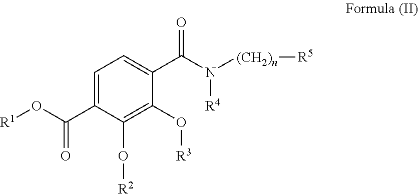

wherein

R¹ is H or alkyl;

R² is H or alkyl;

R³ is H or alkyl;

R⁴ is H or alkyl;

or —O—R³—R⁴—N— together form an optionally substituted, 6 or 7 membered ring;

R⁵ is optionally substituted C₃-C₅ cycloalkyl, optionally substituted lower heterocycloalkyl, optionally substituted aryl or optionally substituted heteroaryl;

where each substituent is independently selected from the group consisting of halogen, —CN, —NO₂, —N₃, ═O, ═S, ═NH, —SO₂, nitroalkyl, amino, dialkylamino, diarylamino, diarylalkylamino, cyanato, isocyanato, thiocyanato, isothiocyanato, guanidinyl, O-carbamyl, N-carbamyl, thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, -L¹-H, -L¹-alkyl, -L¹-substituted alkyl, -L¹-heteroalkyl, -L¹-haloalkyl, -L¹-perhaloalkyl,

-L¹-alkenyl, -L¹-substituted alkenyl, -L¹-heteroalkenyl, -L¹-haloalkenyl, -L¹-perhaloalkenyl, -L¹-alkynyl,
-L¹-substituted alkynyl, -L¹-heteroalkynyl, -L¹-haloalkynyl, -L¹-perhaloalkynyl, -L¹-cycloalkyl, -L¹-substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-unsubstituted aryl, -L¹-heteroaryl and -L¹-substituted heteroaryl;

where -L¹- is a bond, -alkylene-, -heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—,

### —C(S)NH—, —NHC(S)—, —S(O)—, —S(O)₂— or —S(O)NH—;

n is 0, 1 or 2; and a pharmaceutically acceptable salt, pharmaceutically acceptable N-oxide, pharmaceutically active metabolite, pharmaceutically acceptable prodrug, pharmaceutically acceptable solvate thereof.

In a further or alternative embodiment, R¹ is not H. In a further or alternative embodiment, R² and R³ are not methyl. In a further or alternative embodiment, R¹ is not H; and R² and R³ are not methyl. In a further or alternative embodiment, R⁴ is not H. In a further or alternative embodiment, R¹ is not H; R² and R³ are not methyl; and R⁴ is not H. In a further or alternative embodiment, R⁵ is not unsubstituted phenyl. In a further or alternative embodiment, R¹ is not H; R² and R³ are not methyl; R⁴ is not H; and R⁵ is not unsubstituted phenyl. In a further or alternative embodiment, compounds of Formula (I) are with a proviso that when R¹ is H; and R² and R³ are methyl, then R⁴ is not H; and R⁵ is not unsubstituted phenyl.

In a further or alternative embodiment, R¹ is alkyl. In a further or alternative embodiment, R¹ is H, methyl, ethyl, propyl, iso-propyl, butyl, iso-butyl, or tert-butyl. In a further or alternative embodiment, R¹ is H or methyl. In a further or alternative embodiment, R¹ is methyl. In a further or alternative embodiment, R¹ is H. In a further or alternative embodiment, R² is H. In a further or alternative embodiment, R³ is H. In a further or alternative embodiment, R² and R³ are H. In a further or alternative embodiment, R⁴ is H, methyl, ethyl, propyl, iso-propyl, butyl, iso-butyl, or tert-butyl. In a further or alternative embodiment, R⁴ is H or methyl. In a further or alternative embodiment, R⁴ is H. In a further or alternative embodiment, R⁴ is methyl. In a further or alternative embodiment, n is 0. In a further or alternative embodiment, n is 1.

In a further or alternative embodiment, R⁵ is optionally substituted aryl or optionally substituted heteroaryl. In a further or alternative embodiment, R⁵ is substituted aryl or optionally substituted heteroaryl. In a further or alternative embodiment, R⁵ is substituted phenyl or optionally substituted pyridyl. In a further or alternative embodiment, R⁵ is an unsubstituted phenyl or an unsubstituted pyridyl. In a further or alternative embodiment, R⁵ is substituted with at least one group selected from C₁-C₆ alkoxy, C₁-C₆ alkyl, C₁-C₆ haloalkyl, OH, NO₂, or NH₂. In a further or alternative embodiment, R⁵ is selected from the group consisting of:

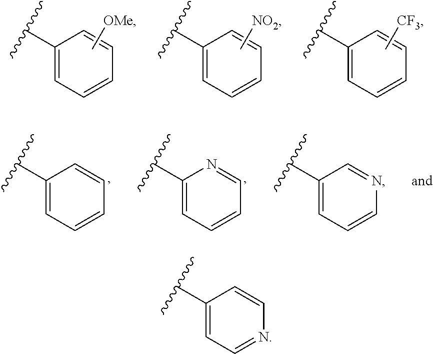

In a further or alternative embodiment, R¹ is alkyl; R²═R³═R⁴═H; R⁵ is substituted phenyl or substituted pyridyl; and n is 0 or 1. In a further or alternative embodiment, R¹ is alkyl; R²═R³═R⁴═H; R⁵ is unsubstituted phenyl or unsubstituted pyridyl; and n is 0 or 1. In a further or alternative embodiment, —O—R³—R⁴—N— together form an optionally substituted, 6 or 7 membered ring.

When —O—R³—R⁴—N— together form an optionally substituted, 6 or 7 membered ring, in a further or alternative embodiment are compounds having the structure of Formula (III):

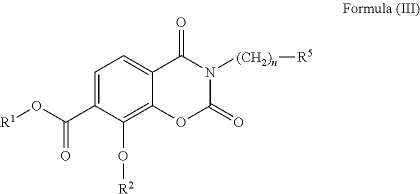

wherein

R¹ is H, alkyl or substituted alkyl;

R² is H, alkyl, substituted alkyl, —C(O)-alkyl, or —C(O)-substituted alkyl;

R⁵ is optionally substituted cycloalkyl, optionally substituted heterocycloalkyl, optionally substituted aryl or optionally substituted heteroaryl;

where each substituent is independently selected from the group consisting of halogen, —CN, —NO₂, —N₃, ═O, ═S, ═NH, —SO₂, nitroalkyl, amino, dialkylamino, diarylamino, diarylalkylamino, cyanato, isocyanato, thiocyanato, isothiocyanato, guanidinyl, O-carbamyl, N-carbamyl, thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, -L¹-H, -L¹-alkyl, -L¹-substituted alkyl, -L¹-heteroalkyl, -L¹-haloalkyl, -L¹-perhaloalkyl,

-L¹-alkenyl, -L¹-substituted alkenyl, -L¹-heteroalkenyl, -L¹-haloalkenyl, -L¹-perhaloalkenyl, -L¹-alkynyl,
-L¹-substituted alkynyl, -L¹-heteroalkynyl, -L¹-haloalkynyl, -L¹-perhaloalkynyl, -L¹-cycloalkyl, -L¹-substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-unsubstituted aryl, -L¹-heteroaryl and -L¹-substituted heteroaryl;

where -L¹- is a bond, -alkylene-, -heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—,

### —C(S)NH—, —NHC(S)—, —S(O)—, —S(O)₂— or —S(O)NH—;

n is 0, 1 or 2; and a pharmaceutically acceptable salt, pharmaceutically acceptable N-oxide, pharmaceutically active metabolite, pharmaceutically acceptable prodrug, pharmaceutically acceptable solvate thereof.

In a further or alternative embodiment, R¹ is alkyl. In a further or alternative embodiment, R¹ is H, methyl, ethyl, propyl, iso-propyl, butyl, iso-butyl, or tert-butyl. In a further or alternative embodiment, R¹ is H or methyl. In a further or alternative embodiment, R¹ is methyl. In a further or alternative embodiment, R¹ is H. In a further or alternative embodiment, R² is H. In a further or alternative embodiment, n is 0. In a further or alternative embodiment, n is 1.

In a further or alternative embodiment, R⁵ is optionally substituted aryl or optionally substituted heteroaryl. In a further or alternative embodiment, R⁵ is substituted aryl or optionally substituted heteroaryl. In a further or alternative embodiment, R⁵ is substituted phenyl or optionally substituted pyridyl. In a further or alternative embodiment, R⁵ is an unsubstituted phenyl or an unsubstituted pyridyl. In a further or alternative embodiment, R⁵ is substituted with at least one group selected from C₁-C₆ alkoxy, C₁-C₆ alkyl, C₁-C₆ haloalkyl, OH, NO₂, or NH₂. In a further or alternative embodiment, R⁵ is selected from the group consisting of:

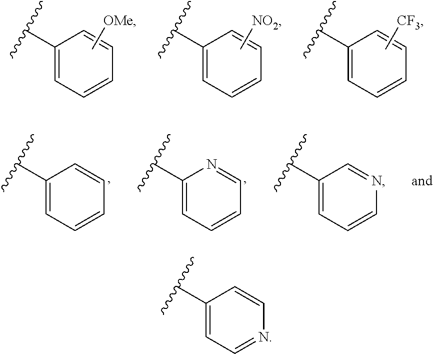

In a further or alternative embodiment, R¹ is alkyl; R² is H; R⁵ is substituted phenyl or optionally substituted pyridyl; and n is 0 or 1.

In another aspect are methods for modulating the activity of an HIV integrase comprising the step of contacting said HIV integrase with at least one compound having the structure of Formula (I), (II) or (III), or their respective pharmaceutically acceptable salts, pharmaceutically active metabolites, pharmaceutically acceptable prodrugs or pharmaceutically acceptable solvates.

In a further or alternative embodiment, R¹ of the compound is alkyl. In a further or alternative embodiment, R² of the compound is H. In a further or alternative embodiment, n of the compound is 0. In a further or alternative embodiment, n of the compound is 1.

In a further or alternative embodiment, R⁵ of the compound is optionally substituted aryl or optionally substituted heteroaryl. In a further or alternative embodiment, R⁵ of the compound is substituted aryl or optionally substituted heteroaryl. In a further or alternative embodiment, R⁵ of the compound is substituted phenyl or optionally substituted pyridyl. In a further or alternative embodiment, R⁵ of the compound is an unsubstituted phenyl or an unsubstituted pyridyl. In a further or alternative embodiment, R⁵ of the compound is substituted with at least one group selected from C₁-C₆ alkoxy, C₁-C₆ alkyl, C₁-C₆ haloalkyl, OH, NO₂, or NH₂. In a further or alternative embodiment, R⁵ of the compound is selected from the group consisting of:

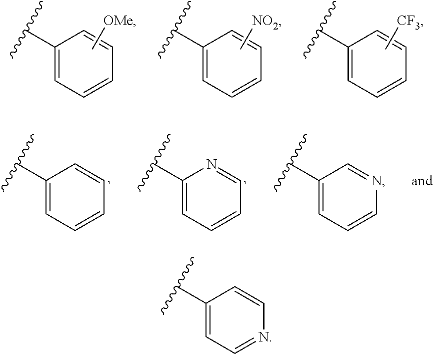

In a further or alternative embodiment, R¹ of the compound is alkyl; R² of the compound is H; R⁵ of the compound is substituted phenyl or optionally substituted pyridyl; and n of the compound is 0 or 1. In a further or alternative embodiment, said compound directly contacts the HIV integrase. In a further or alternative embodiment, said contacting occurs in vitro. In a further or alternative embodiment, said contacting occurs in vivo.

In another aspect are pharmaceutical compositions comprising at least one compound of Formula (I), (II) or (III), or their respective pharmaceutically acceptable salts, pharmaceutically active metabolites, pharmaceutically acceptable prodrugs or pharmaceutically acceptable solvates, in admixture with one or more excipients.

In a further or alternative embodiment, said one or more excipients are for parenteral administration. In a further or alternative embodiment, said one or more excipients are for oral administration.

In another aspect are methods of preventing, inhibiting or ameliorating the pathology and/or symptomology of infection with an immunodeficiency virus in an animal, comprising the step of administering to said animal a therapeutically effective amount of at least one compound of Formula (I), (II) or (III), or their respective pharmaceutically acceptable salts, pharmaceutically active metabolites, pharmaceutically acceptable prodrugs or pharmaceutically acceptable solvates.

In a further or alternative embodiment, R¹ of the compound is alkyl. In a further or alternative embodiment, R¹ of the compound is H, methyl, ethyl, propyl, iso-propyl, butyl, iso-butyl, or tert-butyl. In a further or alternative embodiment, R¹ of the compound is H or methyl. In a further or alternative embodiment, R¹ of the compound is H. In a further or alternative embodiment, R¹ of the compound is methyl. In a further or alternative embodiment, R² of the compound is H. In a further or alternative embodiment, n of the compound is 0. In a further or alternative embodiment, n of the compound is 1.

In a further or alternative embodiment, R⁵ of the compound is optionally substituted aryl or optionally substituted heteroaryl. In a further or alternative embodiment, R⁵ of the compound is substituted aryl or optionally substituted heteroaryl. In a further or alternative embodiment, R⁵ of the compound is substituted phenyl or optionally substituted pyridyl. In a further or alternative embodiment, R⁵ of the compound is an unsubstituted phenyl or an unsubstituted pyridyl. In a further or alternative embodiment, R⁵ of the compound is substituted with at least one group selected from C₁-C₆ alkoxy, C₁-C₆ alkyl, C₁-C₆ haloalkyl, OH, NO₂, or NH₂. In a further or alternative embodiment, R⁵ of the compound is selected from the group consisting of:

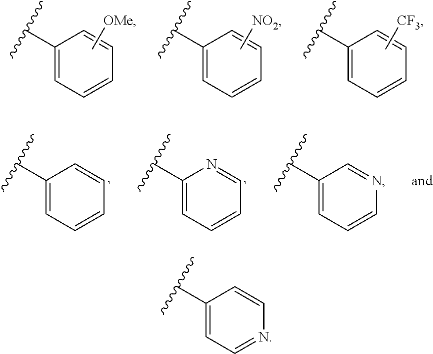

In a further or alternative embodiment, R¹ of the compound is alkyl; R² of the compound is H; R⁵ of the compound is substituted phenyl or optionally substituted pyridyl; and n of the compound is 0 or 1. In a further or alternative embodiment, said compound directly contacts the HIV integrase. In a further or alternative embodiment, said contacting occurs in vitro. In a further or alternative embodiment, said contacting occurs in vivo.

In another aspect are methods of preventing, inhibiting or ameliorating the pathology and/or symptomology of AIDS or infection with HIV in a human, comprising the step of administering to said human a therapeutically effective amount of at least one compound of Formula (I), (II) or (III), or their respective pharmaceutically acceptable salts, pharmaceutically active metabolites, pharmaceutically acceptable prodrugs or pharmaceutically acceptable solvates.

In a further or alternative embodiment, R¹ of the compound is alkyl. In a further or alternative embodiment, R¹ is H, methyl, ethyl, propyl, iso-propyl, butyl, iso-butyl, or tert-butyl. In a further or alternative embodiment, R¹ of the compound is H or methyl. In a further or alternative embodiment, R¹ of the compound is H. In a further or alternative embodiment, R¹ of the compound is methyl. In a further or alternative embodiment, R² of the compound is H. In a further or alternative embodiment, n of the compound is 0. In a further or alternative embodiment, n of the compound is 1.

In a further or alternative embodiment, R⁵ of the compound is optionally substituted aryl or optionally substituted heteroaryl. In a further or alternative embodiment, R⁵ of the compound is substituted aryl or optionally substituted heteroaryl. In a further or alternative embodiment, R⁵ of the compound is substituted phenyl or optionally substituted pyridyl. In a further or alternative embodiment, R⁵ of the compound is an unsubstituted phenyl or an unsubstituted pyridyl. In a further or alternative embodiment, R⁵ of the compound is substituted with at least one group selected from C₁-C₆ alkoxy, C₁-C₆ alkyl, C₁-C₆ haloalkyl, OH, NO₂, or NH₂. In a further or alternative embodiment, R⁵ of the compound is selected from the group consisting of:

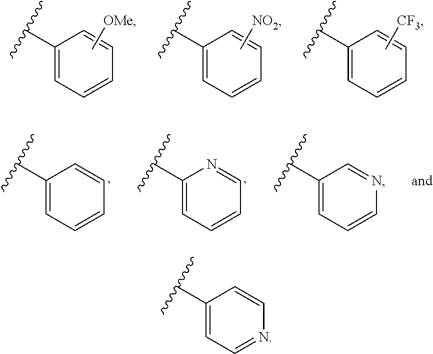

In a further or alternative embodiment, R¹ of the compound is alkyl; R² of the compound is H; R⁵ of the compound is substituted phenyl or optionally substituted pyridyl; and n of the compound is 0 or 1. In a further or alternative embodiment, said compound directly contacts the HIV integrase. In a further or alternative embodiment, said contacting occurs in vitro. In a further or alternative embodiment, said contacting occurs in vivo.

In another aspect are methods of preventing, inhibiting or ameliorating the pathology and/or symptomology of AIDS or infection with HIV in a human, comprising the step of administering to said human a therapeutically effective amount of at least one compound of Formula (I), (II) or (III), or their respective pharmaceutically acceptable salts, pharmaceutically active metabolites, pharmaceutically acceptable prodrugs or pharmaceutically acceptable solvates, as part of a combination therapy.

In a further or alternative embodiment, the method further comprises the step of administration of a therapeutically effective amount of one or more substances, wherein said one or more substances are useful for the prevention, inhibition or amelioration of the pathology and/or symptomology of AIDS or infection with HIV.

In a further or alternative embodiment, the method further comprises the step of administration of a therapeutically effective amount of one or more substances, wherein said one or more substances are therapeutic agents approved by the FDA for the prevention, inhibition or amelioration of the pathology and/or symptomology of AIDS or infection with HIV.

In a further or alternative embodiment, said one or more substances are selected from the group consisting of nucleoside/nucleotide reverse transcriptase inhibitors (NRTI), non-nucleoside reverse transcriptase inhibitors (NNRTI), protease inhibitors (PI), fusion inhibitors and any combination thereof. In a further or alternative embodiment, said one or more substances are selected from the group consisting of Abacavir, Amprenavir, Atazanavir, Delavirdine (DLV), Didanosine (ddI), Efavirenz, Enfuvirtide (T-20), Emtricitabine, Emtricitabine (FTC), Fosamprenavir, Indinavir (IDV), Lamivudine, Lamivudine (3TC), Lopinavir, Nelfinavir, Nevirapine, Ritonavir, Saquinavir, Saquinavir Mesylate, Stavudine (d4T), Tenofovir DF, Viread, Zalcitabine (ddC), Zidovudine and Zidovudine (AZT), and any combination thereof.

In a further or alternative embodiment, said compound is administered simultaneously with said one or more substances. In a further or alternative embodiment, said compound is administered sequentially with said one or more substances. In a further or alternative embodiment, said compound and said one or more substances are administered in the same pharmaceutical composition.

In another aspect is the use of a compound of Formula (I), (II), or (III), in the manufacture of a medicament for treating a disease or condition in an animal in which HIV integrase activity contributes to the pathology and/or symptomology of the disease or condition. In a further or alternative embodiment, said disease or condition is AIDS or infection with HIV.

In another aspect are processes for preparing a compound corresponding to Formula (I), (II), or (III) as HIV integrase inhibitors, their respective N-oxide or other pharmaceutically acceptable derivatives such as prodrug derivatives, or individual isomers and mixture of isomers thereof.

In another aspect are compounds of Formula (I), (II), or (III) for use in a method of treating a disease or condition in an animal in which HIV integrase activity contributes to the pathology and/or symptomology of the disease or condition. In a further or alternative embodiment, said disease or condition is AIDS or infection with HIV.

### INCORPORATION BY REFERENCE

All publications and patent applications mentioned in this specification are herein incorporated by reference to the same extent as if each individual publication or patent application is specifically and individually indicated to be incorporated by reference.

### BRIEF DESCRIPTION OF THE DRAWINGS

FIG. 1 represents the results of molecular modeling showing two possible modes of interaction (1A and 1B) of compound 1 with HIV integrase. Flexible docking is conducted using Glide 2.0 (Schrodinger, Inc, Portland, Oreg., 2002), with protein coordinates taken from the protein databank (pdb code 1FK9).

FIG. 2 represents the results of molecular modeling to dock compound 21 in the integrase active site.

### DETAILED DESCRIPTION OF THE INVENTION

Described are terephthalamates and related compounds that show broad utility, e.g. in inhibiting HIV integrase to thereby treat or prevent AIDS or HIV. Also described are compounds which can be used in combination with other anti-HIV agents such as protease inhibitors, reverse transcriptase inhibitors, fusion inhibitors and the like, to provide a more effective anti-HIV agent.

### Certain Chemical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg, Advanced Organic Chemistry 4^{th} Ed., Vols. A (2000) and B (2001), Plenum Press, New York, N.Y. Unless otherwise indicated, conventional methods of mass spectroscopy, NMR, HPLC, protein chemistry, biochemistry, recombinant DNA techniques and pharmacology, within the skill of the art are employed.

As used in the present specification, the following words and phrases are generally intended to have the meanings as set forth below, except to the extent that the context in which they are used indicates otherwise.

The terms “compound of Formula (I),” “compound of Formula (II),” “compound of Formula (III),” “compound having the structure of Formula (I),” “compound having the structure of Formula (II),” “compound having the structure of Formula (III),” and the like, are intended to encompass the terephthalamate compounds of Formula (I), (II), or (III) as described herein, including their respective pharmaceutically acceptable salts, pharmaceutically active metabolites, pharmaceutically acceptable prodrugs, pharmaceutically acceptable solvates, and pharmaceutically acceptable coordination complexes. In addition, the compounds of Formula (I), (II), or (III) include the individual stereochemical isomers and mixtures thereof, arising from the selection of substituent groups. Some of the compounds of Formula (I), (II) or (III) may contain one or more chiral centers and therefore may exist in enantiomeric and diastereomeric forms. Compounds of Formula (I), (II), or (III) are intended to cover all isomers per se, as well as mixtures of cis and trans isomers, mixtures of diastereomers and racemic mixtures of enantiomers (optical isomers) as well. Further, it is possible using well known techniques to separate the various forms, and some embodiments may feature purified or enriched species of a given enantiomer or diastereomer. Some of the compounds of Formula (I), (II) or (III) may exist in tautomeric forms. Compounds of Formula (I), (II), or (III) are intended to cover all tautomers.

The term “bond” or “single bond” as used herein, refers to a covalent bond between two atoms, either of which may be part of a larger moiety.

The term “moiety” as used herein, alone or in combination, refers to a specific segment or functional group of a molecule. Chemical moieties are often recognized chemical entities embedded in or appended to a molecule.

The term “halo” or “halogen” as used herein, alone or in combination, refers to fluoro, chloro, bromo and iodo.

The term “carbon chain” as used herein, alone or in combination, refers to any alkyl, alkenyl, alkynyl, heteroalkyl, heteroalkenyl or heteroalkynyl group, which is linear, cyclic, or any combination thereof. If the chain is part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining the chain length. If the chain contains heteroatoms as part of the backbone, those atoms are not calculated as part of the carbon chain length.

The term “alkyl” as used herein, alone or in combination, refers to an unsubstituted or substituted, hydrocarbon group and can include straight, branched, cyclic, saturated and/or unsaturated features. Although the alkyl moiety may be an “unsaturated alkyl” moiety, which means that it contains at least one alkene or alkyne moiety, typically, the alkyl moiety is a “saturated alkyl” group, which means that it does not contain any alkene or alkyne moieties. Likewise, although the alkyl moiety may be a cyclic, typically, the alkyl moiety is a non-cyclic group. Thus, most usually, “alkyl” refers to an optionally substituted straight-chain, or optionally substituted branched-chain saturated hydrocarbon monoradical preferably having from about one to about thirty carbon atoms, more preferably from about one to about fifteen carbon atoms and even more preferably from about one to about six carbon atoms. Examples of saturated alkyl radicals include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, sec-butyl, t-butyl, n-pentyl, isopentyl, neopentyl, and n-hexyl, and longer alkyl groups, such as heptyl, and octyl. It should be noted that whenever it appears herein, a numerical range such as “1 to 10” refers to each integer in the given range; e.g., “1 to 10 carbon atoms” or “C₁₋₁₀” or “C₁-C₁₀” means that the alkyl group may consist of 1 carbon atom, 2 carbon atoms, 3 carbon atoms, 4 carbon atoms, 5 carbon atoms, 6 carbon atoms, 7 carbon atoms, 8 carbon atoms, 9 carbon atoms and/or 10 carbon atoms, although the present definition also covers the occurrence of the term “alkyl” where no numerical range is designated.

The term “lower alkyl” as used herein, alone or in combination, refers to an alkyl group, as defined herein, containing fewer carbon atoms, e.g., one containing from one to about six carbon atoms.

The term “substituted alkyl” as used herein, alone or in combination, refers to an alkyl group, as defined herein, in which one or more (up to about five, preferably up to about three) hydrogen atoms is replaced by a substituent independently selected from the substituent group defined herein.

The term “alkylene” as used herein, alone or in combination, refers to a diradical derived from the above-defined monoradical, alkyl. Examples of alkylene diradicals include, but are not limited to, methylene (—CH₂—), ethylene (—CH₂CH₂—), propylene (—CH₂CH₂CH₂—), isopropylene (—CH(CH₃)CH₂—) and the like.

The term “substituted alkylene” as used herein, alone or in combination, refers to a diradical derived from the above-defined monoradical, substituted alkyl.

The term “alkenyl” as used herein, alone or in combination, refers to an optionally substituted straight-chain, or optionally substituted branched-chain hydrocarbon monoradical having from two to about thirty carbon atoms, more preferably from two to about fifteen carbon atoms and even more preferably from two to about six carbon atoms and having one or more carbon-carbon double-bonds. The double bond of an alkenyl group can be unconjugated or conjugated to another unsaturated group. Examples of alkenyl radicals include, but are not limited to, ethenyl or vinyl (—CH═CH₂), 1-propenyl or allyl (—CH₂CH═CH₂), isopropenyl (—C(CH₃)═CH₂), butenyl, 1,3-butadienyl, pentenyl, pentadienyl, hexenyl, hexadienyl, 2-ethylhexenyl, 2-propyl-2-butenyl, 4-(2-methyl-3-butene)-pentenyl, and the like.

The term “lower alkenyl” as used herein, alone or in combination, refers to an alkenyl group, as defined herein, containing fewer carbon atoms, e.g., one containing from two to about six carbon atoms.

The term “substituted alkenyl” as used herein, alone or in combination, refers to an alkenyl group in which one or more (up to about five, preferably up to about three) hydrogen atoms is replaced by a substituent independently selected from the substituent group defined herein.

The term “alkenylene” as used herein, alone or in combination, refers to a diradical derived from the above-defined monoradical, alkenyl. Examples of alkenylene diradicals include, but are not limited to, ethenylene (—CH═CH—), the propenylene isomers (e.g., —CH₂CH═CH— and —C(CH₃)═CH—) and the like.

The term “substituted alkenylene” as used herein, alone or in combination, refers to a diradical derived from the above-defined monoradical, substituted alkenyl.

The term “alkynyl” as used herein, alone or in combination, refers to an optionally substituted straight-chain, or optionally substituted branched-chain hydrocarbon monoradical preferably having from two to about thirty carbon atoms, more preferably from two to about fifteen carbons and even more preferably from two to six carbon atoms and having one or more carbon-carbon triple-bonds. The triple bond of an alkynyl group can be unconjugated or conjugated to another unsaturated group. Examples of alkynyl radicals include, but are not limited to, ethynyl (—C≡CH), 2-propynyl, 2-butynyl, 1,3-butadiynyl, pentynyl, hexynyl, methylpropynyl, 4-methyl-1-butynyl, 4-propyl-2-pentynyl, 4-butyl-2-hexynyl, and the like.

The term “lower alkynyl” as used herein, alone or in combination, refers to an alkynyl group, as defined herein, containing fewer carbon atoms, e.g. one containing from two to about six carbon atoms.

The term “substituted alkynyl” as used herein, alone or in combination, refers to an alkynyl group in which one or more (up to about five, preferably up to about three) hydrogen atoms is replaced by a substituent independently selected from the substituent group defined herein.

The term “alkynylene” as used herein, alone or in combination, refers to a diradical derived from the above-defined monoradical, alkynyl. Examples of alkynylene diradicals include, but are not limited to ethynylene (—C≡C—), propargylene (—CH₂—C≡C—) and the like.

The term “substituted alkynylene” as used herein, alone or in combination, refers to a diradical derived from the above-defined monoradical, substituted alkynyl.

The terms “heteroalkyl,” “heteroalkenyl,” and “heteroalkynyl” as used herein, alone or in combination, refer to optionally substituted alkyl, alkenyl and alkynyl monoradicals respectively, preferably having from two to about thirty atoms, more preferably from two to about fifteen atoms and even more preferably from two to about eight atoms, as described above, and which have one or more skeletal chain atoms selected from an atom other than carbon (i.e. a heteroatom), e.g., oxygen, nitrogen, sulfur, selenium, phosphorus or combinations thereof.

The terms “lower heteroalkyl,” “lower heteroalkenyl,” and “lower heteroalkynyl” as used herein, alone or in combination, refer to the above-defined heteroalkyl, heteroalkenyl and heteroalkynyl groups respectively, containing fewer carbon atoms, e.g., containing from two to about six carbon atoms.

The terms “heteroalkylene,” “heteroalkenylene,” and “heteroalkynylene” as used herein, alone or in combination, refer to diradicals derived from the above-defined heteroalkyl, heteroalkenyl and heteroalkynyl monoradicals, respectively.

The terms “cycloalkyl,” “cycloalkenyl,” and “cycloalkynyl” as used herein, alone or in combination, refer to non-aromatic, optionally substituted, cyclic alkyl, alkenyl and alkynyl monoradicals respectively, including monocyclic, bicyclic, tricyclic, higher multicyclic, polycyclic or multiple condensed ring radicals, wherein each cyclic moiety has from three to about twenty atoms, preferably from three to about fifteen atoms, more preferably from four to about ten atoms. The terms include fused, non-fused, spirocyclic and bridged radicals. A fused cyclic radical may contain from two to four fused rings where the ring of attachment is a cycloalkyl, cycloalkenyl or cycloalkynyl ring, and the other individual rings within the fused radical may be cycloalkyl, cycloalkenyl, cycloalkynyl, heterocycloalkyl, heterocycloalkenyl, heterocycloalkynyl, aromatic, heteroaromatic or any combination thereof. Examples of cycloalkyl groups include, but are not limited to cyclopropyl, cyclobutyl, cyclopentyl, cyclooctyl and the like, or multiple ring structures such as norbornyl, adamantanyl, and the like. A non-limiting example of a cycloalkenyl group is cyclopentadienyl. A non-limiting example of a cycloalkynyl group is cyclopentynyl.

The terms “lower cycloalkyl,” “lower cycloalkenyl,” and “lower cycloalkynyl” as used herein, alone or in combination, refer to the above-defined cycloalkyl, cycloalkenyl and cycloalkynyl groups respectively, containing fewer carbon atoms, e.g., containing from three to about eight carbon atoms.

The terms “heterocycloalkyl,” “heterocycloalkenyl,” and “heterocycloalkynyl” as used herein, alone or in combination, refer to non-aromatic, optionally substituted, cyclic heteroalkyl, heteroalkenyl and heteroalkynyl monoradicals respectively, including monocyclic, bicyclic, tricyclic, higher multicyclic, polycyclic or multiple condensed ring radicals, wherein each cyclic moiety has from three to about twenty atoms, preferably from three to about fifteen atoms, more preferably from four to about ten atoms, and which have one or more cyclic ring atoms selected from an atom other than carbon, e.g., oxygen, nitrogen, sulfur, phosphorous or combinations thereof. The terms include fused, non-fused, spirocyclic and bridged radicals. A fused cyclic radical may contain from two to four fused rings where the ring of attachment is a heterocycloalkyl, heterocycloalkenyl or heterocycloalkynyl ring, and the other individual rings within the fused radical may be cycloalkyl, cycloalkenyl, cycloalkynyl, heterocycloalkyl, heterocycloalkenyl, heterocycloalkynyl, aromatic, heteroaromatic or any combination thereof. Examples of heterocycloalkyl groups include, but are not limited to, pyrrolidinyl, 1,3-dioxalanyl, imidazolidinyl, pyrazolidinyl, piperidinyl, 1,4-dioxanyl, morpholinyl, 1,4-dithianyl, thiomorpholinyl, piperazinyl and the like. Non-limiting examples of heterocycloalkenyl groups include pyrrolinyl, imidazolinyl, pyrazolinyl, pyranyl and the like. A non-limiting example of a fused heterocycloalkyl group is indolinyl.

The terms “lower heterocycloalkyl,” “lower heterocycloalkenyl,” and “lower heterocycloalkynyl” as used herein, alone or in combination, refer to the above-defined heterocycloalkyl, heterocycloalkenyl and heterocycloalkynyl groups respectively, containing fewer ring atoms, e.g., containing from three to about eight atoms.

The terms “haloalkyl,” “haloalkenyl,” and “haloalkynyl” as used herein, alone or in combination, refer to optionally substituted alkyl, alkenyl and alkynyl groups respectively, as defined herein, that are substituted with one or more fluorines, chlorines, bromines or iodines, or combinations thereof. Non-limiting examples of haloalkyl groups are fluoromethyl and bromoethyl. A non-limiting example of a haloalkenyl group is bromoethenyl. A non-limiting example of a haloalkynyl group is chloroethynyl.

The term “perhalo” as used herein, alone or in combination, refers to groups in which all of the H atoms are replaced by fluorines, chlorines, bromines, iodines, or combinations thereof. Thus, as a non-limiting example, the term “perhaloalkyl” refers to an alkyl group, as defined herein, in which all of the H atoms have been replaced by fluorines, chlorines, bromines or iodines, or combinations thereof. A non-limiting example of a perhaloalkyl group is bromochlorofluoromethyl. A non-limiting example of a perhaloalkenyl group is trichloroethenyl. A non-limiting example of a perhaloalkynyl group is tribromopropynyl.

The terms “alicycle” and “alicyclic” as used herein, alone or in combination, refer to any or all of the optionally substituted, saturated partially unsaturated or fully unsaturated, nonaromatic, all-carbon ring, cyclic monoradicals cycloalkyl, cycloalkenyl and cycloalkynyl, as defined herein. These terms include fused, non-fused, spirocyclic, bridged polycyclic or polycyclic ring radicals.

The terms “heterocycle” and “heterocyclic” as used herein, alone or in combination, refer to any or all of the optionally substituted, heteroatom (e.g., oxygen, nitrogen, sulfur, phosphorous or combinations thereof) containing, saturated or unsaturated, nonaromatic ring monoradicals heterocycloalkyl, heterocycloalkenyl and heterocycloalkynyl, as defined herein. These terms include fused and non-fused heterocyclic ring radicals. Examples of heterocyclic groups include, but are not limited to, azepinyl, azepan-2-onyl, azetidinyl, diazepinyl, dihydrofuranyl, dihydropyranyl, dihydrothienyl, dioxanyl, dioxolanyl, 1,4-dioxa-8-aza-spiro[4.5]dec-8-yl, dithianyl, dithiolanyl, homopiperidinyl, imidazolinyl, imidazolidinyl, indolinyl, indolyl, morpholinyl, oxazepinyl, oxepanyl, oxetanyl, oxylanyl, piperidino, piperidyl, piperidinonyl, piperazinyl, pyranyl, pyrazolinyl, pyrazolidinyl, pyrrolidinyl, pyrrolidinonyl, pyrrolinyl, quinolizinyl, thietanyl, tetrahydrofuranyl, tetrahydroquinolyl, tetrahydrothienyl, tetrahydrothiopyranyl, tetrahydropyridinyl, tetrahydropyranyl, thiazepinyl, thiepanyl, thiomorpholinyl, thioranyl, thioxanyl and the like.

The terms “cyclic” and “membered ring” as used herein, alone or in combination, refers to any cyclic structure, including alicyclic, heterocyclic, aromatic, heteroaromatic and polycyclic fused or non-fused ring systems as described herein. The term “membered” is meant to denote the number of skeletal atoms that constitute the ring. Thus, for example, pyridine, pyran, and pyrimidine are six-membered rings and pyrrole, tetrahydrofuran, and thiophene are five-membered rings.

The term “aromatic” as used herein, alone or in combination, refers to a cyclic or polycyclic moiety having a conjugated unsaturated (4n+2)π electron system (where n is a positive integer), sometimes referred to as a delocalized π electron system.

The term “aryl” as used herein, alone or in combination, refers to an optionally substituted, aromatic, cyclic, hydrocarbon monoradical of from six to about twenty ring atoms, preferably from six to about ten carbon atoms and includes fused (or condensed) and non-fused aromatic rings. A fused aromatic ring radical contains from two to four fused rings where the ring of attachment is an aromatic ring, and the other individual rings within the fused ring may be cycloalkyl, cycloalkenyl, cycloalkynyl, heterocycloalkyl, heterocycloalkenyl, heterocycloalkynyl, aromatic, heteroaromatic or any combination thereof. A non-limiting example of a single ring aryl group includes phenyl; a fused ring aryl group includes naphthyl, anthryl, azulenyl; and a non-fused bi-aryl group includes biphenyl.

The term “lower aryl” as used herein, alone or in combination, refers to an aryl group, as defined above, containing fewer skeletal ring carbon atoms, e.g., one containing six to about ten skeletal ring carbons.

The term “arylene” as used herein, alone or in combination, refers to a diradical derived from the above-defined monoradical aryl, (including substituted aryl), and includes for example, groups such as phenylene.

The term “substituted aryl” as used herein, alone or in combination, refers to an aryl group, as defined herein, in which one or more (up to about five, preferably up to about three) hydrogen atoms is replaced by a substituent independently selected from the group defined herein, (except as otherwise constrained by the definition for the aryl substituent).

The term “heteroaryl” as used herein, alone or in combination, refers to an optionally substituted, aromatic, cyclic monoradical containing from about five to about twenty skeletal ring atoms, preferably from five to about ten ring atoms and includes fused (or condensed) and non-fused aromatic rings, and which have one or more (one to ten, preferably about one to about four) ring atoms selected from an atom other than carbon (i.e. a heteroatom) such as, for example, oxygen, nitrogen, sulfur, selenium, phosphorus or combinations thereof. The term heteroaryl includes optionally substituted fused and non-fused heteroaryl radicals having at least one heteroatom. A fused heteroaryl radical may contain from two to four fused rings where the ring of attachment is a heteroaromatic ring and the other individual rings within the fused ring system may be alicyclic, heterocyclic, aromatic, heteroaromatic or any combination thereof. The term heteroaryl also includes fused and non-fused heteroaryls having from five to about twelve skeletal ring atoms, as well as those having from five to about ten skeletal ring atoms. Examples of heteroaryl groups include, but are not limited to, acridinyl, benzo[1,3]dioxole, benzimidazolyl, benzindazolyl, benzoisooxazolyl, benzokisazolyl, benzofuranyl, benzofurazanyl, benzopyranyl, benzothiadiazolyl, benzothiazolyl, benzo[b]thienyl, benzothiophenyl, benzothiopyranyl, benzotriazolyl, benzoxazolyl, carbazolyl, carbolinyl, chromenyl, cinnolinyl, furanyl, furazanyl, furopyridinyl, furyl, imidazolyl, indazolyl, indolyl, indolidinyl, indolizinyl, isobenzofuranyl, isoindolyl, isoxazolyl, isoquinolinyl, isothiazolyl, naphthylidinyl, naphthyridinyl, oxadiazolyl, oxazolyl, phenoxazinyl, phenothiazinyl, phenazinyl, phenoxathiynyl, thianthrenyl, phenathridinyl, phenathrolinyl, phthalazinyl, pteridinyl, purinyl, puteridinyl, pyrazyl, pyrazolyl, pyridyl, pyridinyl, pyridazinyl, pyrazinyl, pyrimidinyl, pyrimidyl, pyrrolyl, quinazolinyl, quinolinyl, quinoxalinyl, tetrazolyl, thiadiazolyl, thiazolyl, thienyl, triazinyl, (1,2,3,)- and (1,2,4)-triazolyl and the like, and their oxides where appropriate, such as for example pyridyl-N-oxide.

The term “lower heteroaryl” as used herein, alone or in combination, refers to a heteroaryl as defined above, containing fewer skeletal ring atoms, e.g., one containing five to about ten skeletal ring atoms.

The term “heteroarylene” as used herein, alone or in combination, refers to a diradical derived from the above-defined monoradical heteroaryl, (including substituted heteroaryl), and is exemplified by the groups 2,6-pyridylene, 2,4-pyridiylene, 1,2-quinolinylene, 1,8-quinolinylene, 1,4-benzofuranylene, 2,5-pyridinylene, 2,5-indolenyl and the like.

The term “substituted heteroaryl” as used herein, alone or in combination, refers to a heteroaryl group, as defined herein, in which one or more (up to about five, preferably up to about three) hydrogen atoms is replaced by a substituent independently selected from the group defined herein, (except as otherwise constrained by the definition for the heteroaryl substituent).

The terms defined above are intended, where applicable, to include their optionally substituted derivatives.

The terms “optional” or “optionally” as used herein, alone or in combination, mean that the subsequently described event or circumstance may or may not occur, but need not occur, and that the description includes instances where the event or circumstance occurs and instances in which it does not.

The term “optionally substituted” as used herein, refers to groups that are substituted or un-substituted. An optionally substituted group may be un-substituted (e.g., —CH₂CH₃), fully substituted (e.g.,

—CF₂CF₃), mono-substituted (e.g., —CH₂CH₂F) or substituted at a level anywhere in-between fully substituted and mono-substituted (e.g., —CH₂CF₃).

The terms “substituents” or “substituted” as used herein, alone or in combination, refer to groups which may be used to replace another group on a molecule. Such groups are known to those of skill in the chemical arts and may include, without limitation, one or more of the following independently selected groups, or designated subsets thereof: halogen, —CN, —NO₂, —N₃, ═O, ═S, ═NH, —SO₂, nitroalkyl, amino, including mono- and di-substituted amino groups, cyanato, isocyanato, thiocyanato, isothiocyanato, guanidinyl, O-carbamyl, N-carbamyl, thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, dialkylamino, diarylamino, diarylalkylamino, -L¹-H, -L¹-alkyl, -L¹-substituted alkyl, -L¹-heteroalkyl, -L¹-haloalkyl, -L¹-perhaloalkyl,

-L¹-alkenyl, -L¹-substituted alkenyl, -L¹-heteroalkenyl, -L¹-haloalkenyl, -L¹-perhaloalkenyl, -L¹-alkynyl,
-L¹-substituted alkynyl, -L¹-heteroalkynyl, -L¹-haloalkynyl, -L¹-perhaloalkynyl, -L¹-cycloalkyl, -L -substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-aryl, -L¹-substituted aryl, -L¹-heteroaryl and -L¹-substituted heteroaryl, wherein -L¹- is a bond, -alkylene-,
-heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—, —C(S)NH—, —NHC(S)—, —S(O)—, S(O)₂— or
—S(O)NH—; all of which may be further optionally substituted, unless otherwise stipulated, and the protected compounds thereof. The protecting groups that may form the protected compounds of the above substituents are known to those of skill in the art and may be found in references such as Greene and Wuts, Protective Groups in Organic Synthesis, 3d Ed., John Wiley & Sons, New York, N.Y. (1999) and Kocienski, Protective Groups, Thieme Verlag, New York, N.Y. (1994) which are incorporated herein by reference in their entirety.

It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, the substituents described for R¹, R², R³, R⁴, R⁵, Rₐ and R_{b} should be generally understood as having a maximum molecular weight of about 1,000 Daltons, and more typically, up to about 500 Daltons, (except in those instances where macromolecular substituents are clearly intended, e.g., polypeptides, polysaccharides, polyethylene glycols, DNA, RNA and the like).

The term “protecting group” as used herein, refers to a chemical moiety which blocks some, or all, reactive moieties and prevents such groups from participating in chemical reactions until the protective group is removed. The procedures and specific groups involved are known to those of skill in the art and can readily be found in reference sources such as Greene and Wuts, Protective Groups in Organic Synthesis, 3^{rd} Ed. (1999) John Wiley & Sons, New York, N.Y., which is incorporated herein by reference in its entirety.

Where chemical groups are specified by their conventional chemical formulas, written from left to right, they equally encompass the chemically identical substituents that would result from writing the structure from right to left; for example, —CH₂O— is equivalent to —OCH₂—.

### Certain Pharmaceutical Terminology

The term “acceptable” with respect to a formulation, composition or ingredient, as used herein, means having no persistent detrimental effect on the general health of the subject being treated.

The term “pharmaceutically acceptable” as used herein, alone or in combination, refers to a material which does not abrogate the biological activity or properties of the compound, and is relatively nontoxic. Thus, a pharmaceutically acceptable component (such as a salt, carrier, excipient or diluent) of a pharmaceutical agent delivery composition containing compounds of Formula (I), (II), or (III) should be (1) compatible with the other ingredients of the delivery composition to deliver the pharmaceutical agent; and (2) where the delivery composition is intended for therapeutic use with an animal (e.g. a human) should not provoke undue adverse side effects, such as toxicity, irritation and allergic response. Side effects are undue when their risk outweighs the benefit provided by the pharmaceutical agent, i.e., the material may be administered to an individual without causing undesirable biological effects or interacting in a deleterious manner with any of the components of the composition in which it is contained.

The term “pharmaceutically acceptable salt” of a compound, as used herein, refers to a salt that is pharmaceutically acceptable. A pharmaceutically acceptable salt is a salt which retains the biological effectiveness and properties of the compounds of Formula (I), (II), or (III) and which are not biologically or otherwise undesirable. In some cases, the compounds of Formula (I), (II), or (III) are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “prodrug” as used herein, refers to a drug or compound in which metabolic processes within the body convert the drug or compound into a pharmacologically active form.

The term “metabolite” as used herein, refers to a derivative of a compound which is formed when the compound is metabolized.

The term “active metabolite” as used herein, refers to a biologically active derivative of a compound that is formed when the compound is metabolized.

The term “metabolized” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

The term “pharmaceutical combination” as used herein, means a product that results from the mixing or combining of more than one active ingredient and includes both fixed and non-fixed combinations of the active ingredients. The term “fixed combination” means that the active ingredients, e.g. at least one compound of Formula (I), (II), or (III) and a co-agent, are both administered to a patient simultaneously, in the form of a single entity or dosage. The term “non-fixed combination” means that the active ingredients, e.g. at least one compound of Formula (I), (II), or (III) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “effective amount” or “therapeutically effective amount” as used herein, refer to a sufficient amount of an agent or compound being administered which will relieve to some extent one or more of the symptoms of the disease or condition being treated, when administered to a mammal in need of such treatment. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. The therapeutically effective amount will vary depending upon the subject and disease condition being treated, the weight and age of the subject, the severity of the disease condition, the particular compound, the dosing regimen to be followed, timing of administration, the manner of administration and the like, all of which can readily be determined by one of ordinary skill in the art. An appropriate effective amount in any individual case may be determined using techniques, such as a dose escalation study.

The terms “enhance” or “enhancing” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount” as used herein, refers to an amount adequate to enhance the effect of another therapeutic agent in a desired system.

The term “modulate” or “modulating” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist and an antagonist.

The terms “co-administration” and the like, as used herein, are meant to encompass administration of the selected therapeutic agents to a single patient, and are intended to include treatment regimens in which the agents are administered by the same or different route of administration or at the same or different time.

The term “pharmaceutical composition” as used herein, refers to a mixture of an active compound with other chemical components, such as carriers, stabilizers, diluents, dispersing agents, suspending agents, thickening agents, and/or excipients.

The terms “carrier,” “pharmaceutically acceptable carrier,” or “pharmaceutically acceptable excipient” as used herein, refer to relatively nontoxic chemical compounds or agents that facilitate the incorporation of a compound into cells or tissues. They include any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating,” or “treatment” as used herein, include at least partially alleviating, abating or ameliorating a disease or condition symptoms, at least partially preventing additional symptoms, ameliorating or preventing the underlying metabolic causes of symptoms, at least partially inhibiting the disease or condition, e.g., arresting the development of the disease or condition, at least partially relieving the disease or condition, at least partially causing regression of the disease or condition, at least partially relieving a condition caused by the disease or condition, or at least partially stopping the symptoms of the disease or condition. Thus any treatment of a disease in a mammal should provide at least a partial therapeutic or prophylactic effect, including any, all or a combination of the following:

a) preventing the onset of disease, that is, causing the clinical symptoms of the disease not to develop;
b) delaying the onset of disease, that is, causing the clinical symptoms of the disease to develop at a later time;
c) reducing the severity of the onset of disease, that is causing the clinical symptoms of the disease to develop less severely;
c) relieving an ongoing disease, that is, causing the regression of clinical symptoms;
e) arresting an ongoing disease, that is, causing the elimination of clinical symptoms; and/or
d) enhancing normal physiological functioning.

The terms “kit” and “article of manufacture” are used as synonyms.

### Biological Activity and Utility

Described are terephthalamates and related compounds that show broad utility, e.g. in inhibiting HIV integrase to thereby treat or prevent AIDS or HIV. The compounds of Formula (I), (II) or (III) may also be used in combination with other anti-HIV agents such as protease inhibitors, reverse transcriptase inhibitors, fusion inhibitors and the like, to provide a more effective anti-HIV agent.

Human Immunodeficiency Virus (HIV), a retrovirus, is the causative agent of Acquired Immunodeficiency Syndrome (AIDS). HIV targets CD4⁺ cells, (such as helper T cells, macrophages and dendritic cells) and destroys these immunocompetent cells to cause immunodeficiency. Accordingly, a pharmaceutical agent that eradicates HIV in a living organism or suppresses its growth will be effective for the treatment or prophylaxis of AIDS.

The HIV virus comprises an inner core (or capsid), covered with an envelope protein. The inner core contains three enzymes required for HIV replication called reverse transcriptase, integrase and protease, along with HIV's genetic material, which consists of two identical strands of RNA. In general, HIV has nine genes (compared to more than 500 genes in a bacterium, and around 20,000-25,000 in a human). Three of the HIV genes, gag, pol and env, contain information needed to make structural proteins for new virus particles. The other six genes, tat, rev, nef, vif, vpr and vpu, code for proteins that control the ability of HIV to infect a cell, produce new copies of virus, or cause disease.

In general, HIV can only replicate inside human cells. The process typically begins when a virus particle encounters a potential host cell and the HIV viral envelope fuses with the host cell membrane. The contents of the HIV particle, an RNA-integrase complex, are then released into the cell cytoplasm. Once inside the cell, the HIV enzyme reverse transcriptase converts the viral RNA into full length double stranded DNA, which is compatible with human genetic material. This DNA is transported to the cell's nucleus, where it is spliced into the human DNA by the HIV enzyme integrase. Once integrated, the HIV DNA is known as provirus. HIV provirus may lie dormant within a cell for a long time. But when the cell becomes activated, it treats HIV genes in much the same way as human genes. First it converts them into messenger RNA (using human enzymes). Then the messenger RNA is transported outside the nucleus, and is used as a blueprint for producing new HIV proteins and enzymes. Among the strands of messenger RNA produced by the cell are complete copies of HIV genetic material. These gather together with newly made HIV proteins and enzymes to form new viral particles, which are then released from the cell. The enzyme protease plays a vital role at this stage of HIV's life cycle by chopping up long strands of protein into smaller pieces, which are used to construct mature viral cores. The newly matured HIV particles are ready to infect another cell and begin the replication process all over again. In this way the virus quickly spreads through the human body.

Thus, various viral enzymes are essential for HIV replication. These enzymes have drawn much attention as targets for antiviral agents, and several anti-HIV agents have been developed. To date, all FDA-approved anti-HIV drugs are based on the inhibition of HIV-1 protease (e.g. indinavir, nelfinavir), reverse transcriptase (e.g. zidovudine, didanosine, lamivudine), or viral entry. In addition, multiple drug combination therapies have been employed. For example, a combined use of two reverse transcriptase inhibitors (zidovudine and didanosine), and a combined use of two reverse transcriptase inhibitors (zidovudine and lamivudine) with a protease inhibitor (nelfinavir) have been clinically applied. Such multiple drug combination therapy is becoming a mainstream of AIDS therapy.

However, some of these drugs are known to cause side effects such as liver function failure, or CNS disorders (e.g., vertigo). In addition, development of resistance to these drugs is become an increasing challenge for the management of AIDS. See Imamichi, Curr. Pharm. Des. (2004) 10: 4039; De Clercq, J. Med. Chem. (2005) 48: 1297. Even worse, emergence of HIV strains that show multiple drug resistance to a multiple drug combination therapy has been observed.

Thus, there is an urgent need for novel, safe anti-HIV drugs with new mechanism of actions. HIV integrase is an enzyme critical for the incorporation of HIV DNA into host chromosomal DNA. See Esposito et al, Adv. Virus Res. (1999) 52: 319; Dyda et al, Science (1994) 266: 1981. While HIV integrase has been recognized as a promising anti-HIV target for more than a decade, no HIV integrase inhibitors have yet received FDA approval. See Pommier et al, Nat. Rev. Drug Discovery (2005) 4: 236; Anthony, Curr. Top. Med. Chem. (2004) 4: 979; Johnson et al, Curr. Top. Med. Chem. (2004) 4: 1059; Pommier et al, Nature Rev. Drug Discovery (2005) 4: 236 and Nair, Frontiers Med. Chem. (2005) 2: 3. At least five small molecule HIV integrase inhibitors enter clinical trails (one is subsequently halted during phase II). The details of these and other HIV integrase inhibitors are the subject of a review by Cotelle in Recent Patents on Anti-infective Drug Discovery, (2006) 1: 1-15, which is herein incorporated in its entirety. One major challenge in this field is to identify compounds that selectively inhibit HIV integrase with anti-HIV activity. Described are a series of novel HIV integrase inhibitors that are also potent inhibitors for HIV replication.

### Processes for Making Compounds of Formula (I), (II), or (III)

The compounds of Formula (I), (II) or (III) as described herein may be synthesized using standard synthetic techniques known to those of skill in the art or using methods known in the art in combination with methods described herein. In addition, solvents, temperatures and other reaction conditions presented herein may vary according to the practice and knowledge of those of skill in the art.

The starting materials used for the synthesis of the compounds of Formula (I), (II) or (III) as described herein, can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, Advanced Organic Chemistry 4^{th} Ed. (1992) John Wiley & Sons, New York, N.Y.; Carey and Sundberg, Advanced Organic Chemistry 4^{th} Ed., Vols. A (2000) and B (2001) Plenum Press, New York, N.Y. and Greene and Wuts, Protective Groups in Organic Synthesis, 3rd Ed. (1999) John Wiley & Sons, New York, N.Y., (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Formation of Covalent Linkages by Reaction of an Electrophile with a Nucleophile

The compounds described herein can be modified using various electrophiles or nucleophiles to form new functional groups or substituents. Table 1 entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophilic groups and nucleophilic groups.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

Use of Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

In some embodiments, blocking/protecting groups may be selected from:

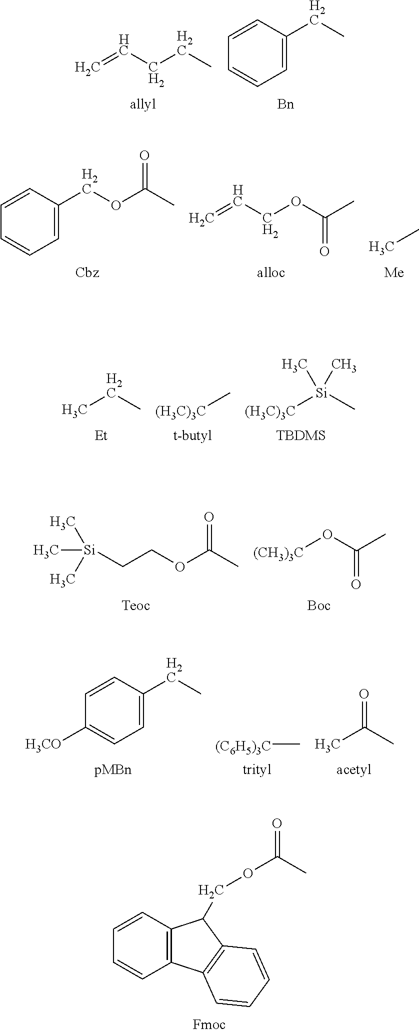

Other protecting groups, plus a detailed description of techniques applicable to the creation of protecting groups and their removal are described in Greene and Wuts, Protective Groups in Organic Synthesis, 3rd Ed. (1999) John Wiley & Sons, New York, N.Y., and Kocienski, Protective Groups (1994) Thieme Verlag, New York, N.Y., which are incorporated herein by reference in their entirety.

In one embodiment, N-benzyl-hydroxybenzamide derivatives are prepared from the corresponding carboxylic acids using HATU as the coupling reagent followed by removal of the methoxy groups using boron tribormide, according to Scheme 1.

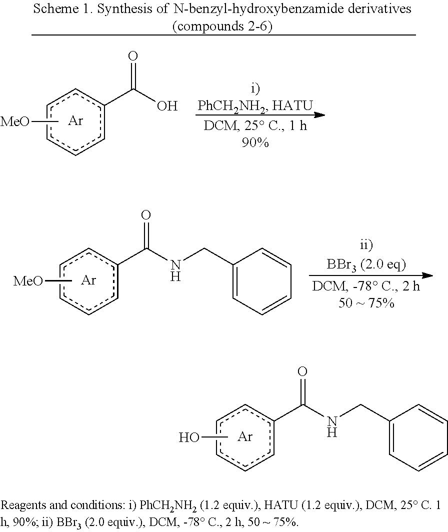

In a further or alternative embodiment, methyl 4-(benzylcarbamoyl)-2,3-dihydroxybenzoate derivatives, are prepared. Synthesis of 2,3-Dihydroxy-terephthalic acid monomethyl ester from catechol is reported by Chen et al, Org. Prep. Proced. Int. (1999) 31: 106 and Gramer et al, Org. Lett. (2001) 3: 2827, which are both incorporated by reference in their entireties. Because Scheme 1 may involve high pressure and long reaction times, a more practical, alternative route is also established as shown in Scheme 2. Starting from catechol, the two hydroxy groups are first protected as MOM ethers. Lithiation with n-butyl lithium at 0° C., followed by the addition of carbon dioxide gives the corresponding bis-carboxylic acids as lithium salts. Treatment with trimethyl silyl chloride in refluxing methanol furnishes the dimethyl and monomethyl esters in a 2:1 ratio and a combined 80% yield. The diester is easily converted to the monoacid using sodium bicarbonate. Treatment of the monoacid with excess thionyl chloride followed by various benzyl amines provides the desired products. The N methyl compound (compound 19) and phenyl compound (compound 20) are prepared using the same chemistry.

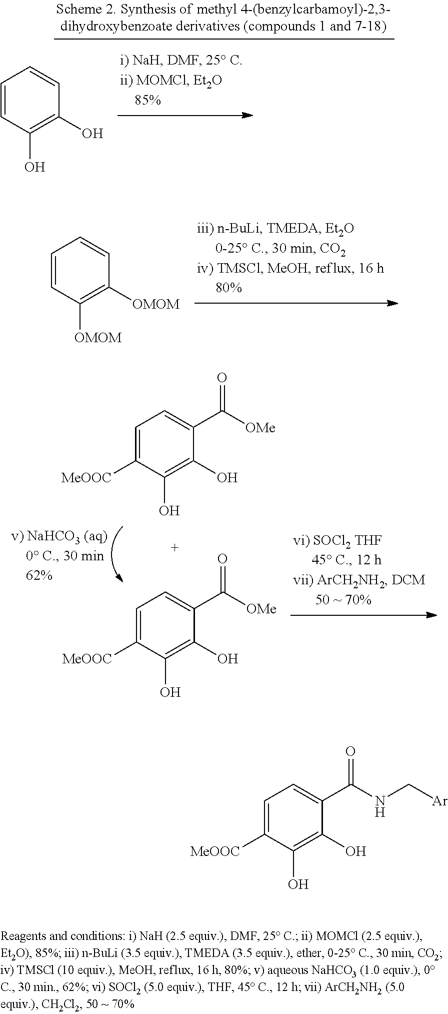

In a further or alternative embodiment, a rigid compound 21, in which the amide N and its neighboring hydroxy oxygen are connected via a carbonyl group is prepared according to Scheme 3.

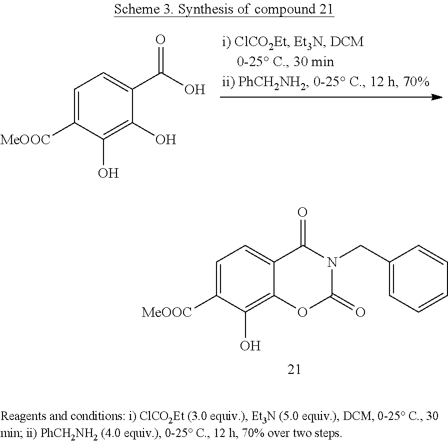

### Further Forms of Compounds

Compounds of Formula (I), (II), or (III) can be prepared as pharmaceutically acceptable salts formed when an acidic proton present in the parent compound either is replaced by a metal ion, for example an alkali metal ion, an alkaline earth ion, or an aluminum ion; or coordinates with an organic base. In addition, the salt forms of the disclosed compounds can be prepared using salts of the starting materials or intermediates.

Compounds of Formula (I), (II) or (III) can be prepared as pharmaceutically acceptable acid addition salts (which are a type of pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Alternatively, compounds of Formula (I), (II) or (III) can be prepared as pharmaceutically acceptable base addition salts (which are a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds of Formula (I), (II) or (III) can be conveniently prepared or formed during the processes described herein. By way of example only, hydrates of compounds of Formula (I), (II) or (III) can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Compounds of Formula (I), (II) or (III) include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compounds of Formula (I), (II), or (III) can comprise nitrogen containing heterocycles or nitrogen containing heteroaryls, such as, for example pyridine groups. It should be understood that compounds of Formula (I), (II), or (III) may exist in their unoxidized for or their oxidized for, i.e. as their N-oxides. The unoxidized forms can be prepared from N-oxides of compounds of Formula (I), (II) or (III) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

Compounds of Formula (I), (II), or (III) can be prepared as prodrugs. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of Formula (I), (II), or (III) which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See for example Fedorak et al, Am. J. Physiol. (1995) 269, G210-218; McLoed et al, Gastroenterol (1994) 106, 405-413; Hochhaus et al, Biomed. Chrom, (1992) 6, 283-286; Larsen and Bundgaard, Int. J. Pharmaceutics (1987) 37, 87; Larsen et al, Int. J. Pharmaceutics (1988) 47, 103; Sinkula et al, J. Pharm. Sci. (1975) 64, 181-210; Higuchi and Stella, Pro-drugs as Novel Delivery Systems, Vol. 14 of the A.C.S. Symposium Series; and Roche, Bioreversible Carriers in Drug Design (1987) American Pharmaceutical Association and Pergamon Press, all incorporated herein in their entirety.

Additionally, prodrug derivatives of compounds of Formula (I), (II) or (III) can be prepared by methods known to those of ordinary skill in the art (for further details see for example Saulnier et al, Bioorg. and Med. Chem. Lett. (1994) 4, p. 1985). By way of example only, appropriate prodrugs can be prepared by reacting a non-derivatized compound of Formula (I), (II), or (III) with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

Sites on the aromatic ring portion of compounds of Formula (I), (II) or (III) can be susceptible to various metabolic reactions, therefore incorporation of appropriate substituents on the aromatic ring structures, such as, by way of example only, halogens can reduce, minimize or eliminate this metabolic pathway.

In other embodiments, the compounds described herein may be labeled isotopically (e.g. with a radioisotope) or by another other means, including, but not limited to, the use of chromophores or fluorescent moieties, bioluminescent labels, or chemiluminescent labels. The compounds of Formula (I), (II) or (III) may possess one or more chiral centers and each center may exist in the R or S configuration. The compounds presented herein include all diastereomeric, enantiomeric, and epimeric forms as well as the appropriate mixtures thereof. Compounds of Formula (I), (II) or (III) can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds described herein, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chiral chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jacques, Collet and Wilen, Enantiomers, Racemates and Resolutions (1981) John Wiley & Sons, New York, N.Y., herein incorporated by reference in its entirety.

Additionally, the compounds and methods provided herein may exist as geometric isomers. The compounds and methods provided herein include all cis, trans, syn, anti, entgegen (E), and zusammen (Z) isomers as well as the appropriate mixtures thereof. In some situations, compounds may exist as tautomers. All tautomers are included within the formulas described herein are provided by compounds and methods herein. In additional embodiments of the compounds and methods provided herein, mixtures of enantiomers and/or diastereoisomers, resulting from a single preparative step, combination, or interconversion may also be useful for the applications described herein.

### Pharmaceutical Composition/Formulation/Administration

A pharmaceutical composition, as used herein, refers to a mixture of at least one compound Formula (I), (II), or (III) with other chemical components, such as carriers, stabilizers, diluents, dispersing agents, suspending agents, thickening agents, and/or excipients. The pharmaceutical composition facilitates administration of the compound to an organism. Pharmaceutical compositions containing at least one compound of Formula (I), (II), or (III) can be administered in therapeutically effective amounts as pharmaceutical compositions by any conventional form and route known in the art including, but not limited to: intravenous, oral, rectal, aerosol, parenteral, ophthalmic, pulmonary, transdermal, vaginal, otic, nasal, and topical administration.

One may administer pharmaceutical compositions in a local rather than systemic manner, for example, via injection of the compound directly into an organ, often in a depot or sustained release formulation. Furthermore, one may administer pharmaceutical compositions containing at least one compound of Formula (I), (II), or (III) in a targeted drug delivery system, for example, in a liposome coated with organ-specific antibody. The liposomes will be targeted to and taken up selectively by the organ. In addition, pharmaceutical compositions containing at least one compound of Formula (I), (II), or (III) may be provided in the form of rapid release formulations, in the form of extended release formulations, or in the form of intermediate release formulations.

For oral administration, compounds of Formula (I), (II) or (III) can readily be formulated by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner. Parental injections may involve for bolus injection or continuous infusion. The pharmaceutical compositions of Formula (I), (II), or (III) may be in a form suitable for parenteral injection as sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredients may be in powder form for constitution with a suitable vehicle, e.g., sterile pyrogen-free water, before use.

The compounds of Formula (I), (II) or (III) can be administered topically and can be formulated into a variety of topically administrable compositions, such as solutions, suspensions, lotions, gels, pastes, medicated sticks, balms, creams or ointments. Such pharmaceutical compositions can contain solubilizers, stabilizers, tonicity enhancing agents, buffers and preservatives.

Formulations suitable for transdermal administration of the compounds of Formula (I), (II) or (III) may employ transdermal delivery devices or transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds of Formula (I), (II) or (III) can be accomplished by means of iontophoretic patches and the like. Additionally, transdermal patches can provide controlled delivery of the compounds of Formula (I), (II) or (III). The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, the compounds of Formula (I), (II) or (III) may be in a form such as an aerosol, a mist or a powder. Pharmaceutical compositions comprising at least one compound of Formula (I), (II), or (III) can be conveniently delivered in the form of an aerosol spray presentation from pressurized packs or nebulisers, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

The compounds of Formula (I), (II) or (III) may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In practicing the methods of treatment or use provided herein, therapeutically effective amounts of compounds of Formula (I), (II) or (III) provided herein are administered in pharmaceutical compositions to a mammal having a disease or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising at least one compound of Formula (I), (II), or (III) may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and at least one compound of Formula (I), (II), or (III) as described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A summary of pharmaceutical compositions described herein may be found, for example, in Remington, The Science and Practice of Pharmacy, 19^{th} Ed. (1995) Mack Publishing Company, Easton, Pa.; Hoover, Remington's Pharmaceutical Sciences (1975) Mack Publishing Company, Easton, Pa.; Liberman and Lachman, Pharmaceutical Dosage Forms (1980) Marcel Decker, New York, N.Y.; and Lippincott, Williams & Wilkins, Pharmaceutical Dosage Forms and Drug Delivery Systems, 7^{th} Ed. (1999) all of which are herein incorporated by reference in their entirety.

### Methods of Administration and Treatment Methods

Compounds of Formula (I), (II) or (III) can be used in the preparation of medicaments for the treatment of diseases or conditions in which HIV integrase activity contributes to the pathology and/or symptomology of the disease, most typically in the treatment of AIDS or infection with HIV. A method for treating AIDS or infection with HIV in a subject in need of such treatment, involves administration of pharmaceutical compositions containing at least one compound of Formula (I), (II), or (III), or a pharmaceutically acceptable salt, pharmaceutically active metabolite, pharmaceutically acceptable prodrug, or pharmaceutically acceptable solvate thereof, in therapeutically effective amounts to said subject.

Compositions containing at least one compound of Formula (I), (II) or (III), as described herein can be administered for prophylactic and/or therapeutic treatments. In therapeutic applications, the compositions are administered to a patient already suffering from AIDS or infected with HIV, in an amount sufficient to cure or at least partially arrest the symptoms of the disease or condition. Amounts effective for this use will depend on many factors, including but not limited to the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease or condition is retained. Patients can, however, require intermittent treatment on a long-term basis upon any recurrence of symptoms.

In certain instances, it may be appropriate to administer therapeutically effective amounts of at least one of the compounds described herein (or a pharmaceutically acceptable salts, pharmaceutically active metabolites, pharmaceutically acceptable prodrugs, and pharmaceutically acceptable solvates thereof) in combination with another therapeutic agent. Indeed, combination therapy, comprising at least three anti-HIV drugs, has become the standard treatment of AIDS. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit.

The overall benefit experienced by the patient may be additive of the two therapeutic agents or the patient may experience a synergistic benefit. For example, synergistic effects can occur with compounds of Formula (I), (II) or (III) and other substances used in the treatment of HIV and AIDS. Most typically, at least one compounds of Formula (I), (II), or (III) described herein would be co-administered with another anti HIV or anti AIDS therapeutic. Most preferably the other therapeutic agent or agents would be approved by the FDA for use in HIV or AIDS prophylaxis or treatment. Such therapeutic agents could work by any of the existing mechanisms of action known to treat HIV/AIDS, such as nucleoside/nucleotide reverse transcriptase inhibitors (NRTI), non-nucleoside reverse transcriptase inhibitors (NNRTI), protease inhibitors (PI), fusion inhibitors or by some other mechanism. Drugs for the prophylaxis or treatment of HIV or AIDS include, but are not limited to Abacavir, AGENERASE®, Amprenavir, Atazanavir, COMBIVIR®, CRIXIVAN®, Delavirdine (DLV), Didanosine (ddI), Efavirenz, Enfuvirtide (T-20), Emtricitabine, Emtricitabine (FTC), EMTRIVA®, EPIVIR®, EPZICOM™, FORTORASE®, FORTOVASE®, Fosamprenavir, FUZEON®, HIVID®, HIVID® ddc, Indinavir (IDV), INVIRASE®, KALETRA®, Lamivudine, Lamivudine (3TC), LEXIVA®, Lopinavir, Nelfinavir, Nevirapine, NORVIR®, RESCRIPTOR®, RETROVIR®, REYATAZ®, Ritonavir, Saquinavir, Saquinavir Mesylate, Stavudine (d4T), SUSTIVA®, Tenofovir DF, TRIZIVIR®, TRUVADA®, VIDEX®, VIRACEPT®, VIRAMUNE®, Viread, Zalcitabine (ddC), ZERIT®, Zidovudine, Zidovudine (AZT), ZIAGEN®, however any combination therapy including at least one compound of Formula (I), (II), or (III) with any other therapeutic agent that would provide a beneficial effect to the patient is also contemplated.

Where the compounds described herein are administered in conjunction with other therapies, dosages of the co-administered compounds will of course vary depending on many factors, including, but not limited to the type of co-drug employed, the specific drug employed, the disease or condition being treated, the severity of the disease or condition being treated and so forth. In addition, when co-administered with one or more pharmaceutically active agents, the compound provided herein may be administered either simultaneously with the pharmaceutically active agent(s), or sequentially. If administered sequentially, the attending physician will decide on the appropriate sequence of administration in combination with the pharmaceutically active agent(s).

In any case, the multiple therapeutic agents (at least one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; multiple therapeutic combinations are also envisioned.

The compounds of Formula (I), (II) or (III) and any combination therapies comprising at least one compound of Formula (I), (II), or (III) can be administered before, during or after exposure to the HIV virus, and the timing of administering the composition can vary. Thus, for example, the compounds can be used prophylactically and can be administered to subjects that may not be infected with the HIV virus, but who have been exposed to the virus or who suspect they may have been exposed to the virus. By way of an example, a health care worker (e.g. doctor, nurse, laboratory technician) may be accidentally exposed (e.g. by needle stick or sample spill) to a sample that may or may not contain HIV. At least one compound of Formula (I), (II), or (III) would be administered in order to prevent or lower the risk of infection. Similarly, in some embodiments, the compounds of Formula (I), (II), or (III) described herein may be used prophylactically for subjects that have been exposed or suspect they may have been exposed to the HIV virus (for example by sexual contact, sharing of needles, childbirth) but that may not yet have developed symptoms of the disease. In other embodiments, the compounds of Formula (I), (II) or (III) can be used prophylactically and can be administered continuously to subjects with a propensity to conditions or diseases in order to prevent the occurrence of the disease or condition. The compounds and compositions can be administered to a subject during or as soon as possible after the onset of the symptoms.

The administration of the compounds can be initiated within the first 48 hours of exposure to the virus or the onset of the symptoms, preferably within the first 48 hours of exposure to the virus or the onset of the symptoms, and more preferably within the first 6 hours of exposure to the virus or the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after exposure, or suspected exposure to the virus, or the onset of a disease or condition is detected or suspected, and for a length of time necessary for the treatment of the disease, such as, for example, from about 1 week to about 1 year. The length of treatment can vary for each subject, and the length can be determined using the known criteria. For example, at least one compound or a formulation comprising at least one compound can be administered for at least 2 weeks, about 1 month and up to about fifteen years.

In a further or alternative embodiment, the pharmaceutical compositions described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, the daily dosages appropriate for the compounds of Formula (I), (II) or (III) as described herein are from about 0.01 to 5 mg/kg per body weight. An indicated daily dosage in the larger mammal, including, but not limited to, humans, is in the range from about 0.5 mg to about 100 mg, conveniently administered in divided doses, including, but not limited to, up to four times a day or in retard form. Suitable unit dosage forms for oral administration comprise from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

### EXAMPLES

The following examples provide illustrative methods for making and testing the effectiveness and safety of the compounds of Formula (I), (II) or (III). These examples are provided for illustrative purposes only and not to limit the scope of the claims provided herein. All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the claims. All such similar substitutes and modifications apparent to those skilled in the art are deemed to be within the spirit, scope and concept of the appended claims.

### Example 1

### Synthesis of methyl 4-(benzylcarbamoyl)-2,3-dihydroxybenzoate (compound 1)

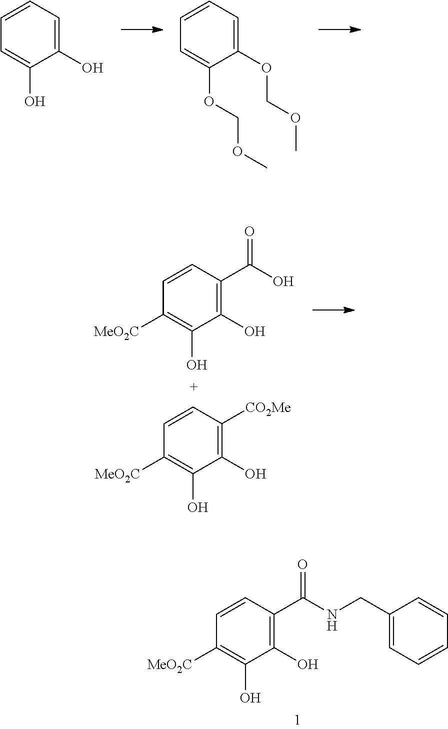

### Example 1a

### Preparation of 1,2-Bis-methoxymethoxy-benzene

NaH (60% in mineral oil, 11 g, 0.25 mol) is added, in three portions, to a solution of catechol (11 g, 0.1 mol) in DMF/ether (1:1, 800 ml). The reaction mixture is shaken for 30 min, and then methoxymethyl chloride (MOM-Cl; 19 ml, 0.25 mol) is added. The mixture is shaken for a further 2 hours at room temperature and then quenched, at 0° C., by slow addition of water (500 ml). The mixture is extracted with hexanes (3×500 ml), and the combined organic layers are washed with water and brine respectively, dried over Na₂SO₄, and concentrated. The residue is purified by silica gel chromatography to give 18 g of product (90%); ¹H NMR (400 MHz, CDCl₃) δ 7.16 (m, 2H), 6.95 (m, 2H), 5.24 (s, 4H), 3.52 (s, 6H).

### Example 1b

### Preparation of 2,3-Dihydroxy-terephthalic acid monomethyl and dimethyl esters

n-BuLi (136.4 mmol) is slowly added to a solution of 1,2-Bis-methoxymethoxy-benzene (9 g, 45.5 mmol) and tetramethylethylenediamine (TMEDA; 21 ml, 136.5 mmol) in ether (500 m), at 0° C. The mixture is allowed to warm to room temperature and stirred for 30 min. Dry CO₂ is bubbled through the reaction mixture for 1 hour. The ether is removed under vacuum and the resulting yellow residue is suspended in anhydrous methanol (300 ml). Chlorotrimethylsilane (160 ml) is added at room temperature. The mixture is refluxed overnight, cooled to 0° C. and water (300 ml) added. The precipitate is isolated by filtration and recrystallized from methanol and water to afford 2,3-dihydroxy-terephthalic acid dimethyl ester (5.7 g, 55%) and 2,3-dihydroxy-terephthalic acid monomethyl ester (2.4 g, 25%); 2,3-Dihydroxy-terephthalic acid dimethyl ester: ¹H NMR (400 MHz, DMSO-d6) δ 10.5 (br, s, 1H), 7.27 (s, 2H), 3.90 (s, 6H), MS m/z 227 [M+H]⁺; 2,3-Dihydroxy-terephthalic acid monomethyl ester: ¹H NMR (400 MHz, DMSO-d6) δ 9.92 (s, 1H), 7.17 (d, 1H), 6.91 (d, 1H), 3.85 (s, 3H), MS m/z 213 [M+H]⁺.

### Example 1c

### Preparation of Methyl 4-(benzylcarbamoyl)-2,3-dihydroxybenzoate (compound 1)

Thioyl chloride (1.5 ml) is added to a solution of 2,3-dihydroxy-terephthalic acid monomethyl ester (300 mg, 1.42 mmol) in anhydrous THF (18 ml). The mixture is stirred at 45° C. for 12 hours, after which time THF and excess SOCl₂ are removed under vacuum. The residue is diluted with CH₂Cl₂ (10 ml) and is slowly added to a solution of benzyl amine in CH₂Cl₂ (15 ml) at 0° C. The reaction mixture is allowed to warm to room temperature for 2 hours, followed by the addition of water (10 ml). Solvents are removed under vacuum and the resulting residue extracted with EtOAc. The combined organic layers are washed with brine, dried over Na₂SO₄, and concentrated under vacuum to give the crude product which is purified by chromatography on silica to afford the title product (220 mg, 52%); ¹H NMR (400 MHz, CDCl₃) δ 11.2 (s, 1H), 10.9 (s, 1H), 7.37 (m, 5H), 7.32 (d, 1H), 7.03 (d, 1H), 6.95 (br, 1H), 4.66 (d, 2H), 3.98 (s, 3H), MS m/z 302 [M+H]⁺.

### Example 2

### Synthesis of N-benzyl-2,3-dihydroxybenzamide (compound 2)

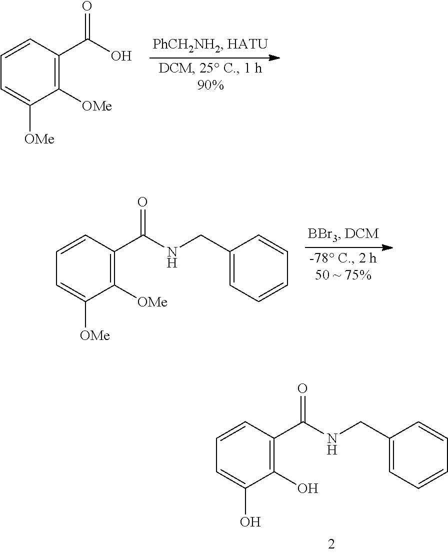

### Example 2a

### Preparation of N-benzyl-2,3-dimethoxybenzamide

2,3-dimethoxybenzoic acid (1 equivalent), benzylamine (1.2 equivalent) and HATU (1.2 equivalent) are reacted in dichloromethane for 1 hour at room temperature to give N-benzyl-2,3-dimethoxybenzamide (90%).

### Example 2b

### Preparation of N-benzyl-2,3-dihydroxybenzamide (compound 2)

N-benzyl-2,3-dimethoxybenzamide (1 equivalent) and BBr₃ (2 equivalent) re reacted in dichloromethane for 2 hours at −78° C. to give N-benzyl-2,3-dihydroxybenzamide (50-75%).

### Example 3

### Synthesis of N-benzyl-3-hydroxybenzamide (compound 3)

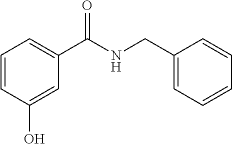

The title compound is prepared by the same method as for compound 2 (example 2), using 3-methoxybenzoic acid in place of 2,3-dimethoxybenzoic acid.

### Example 4

### Synthesis of N-benzyl-2-hydroxybenzamide (compound 4)

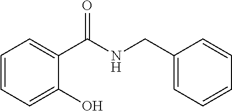

The title compound is prepared by the same method as for compound 2 (example 2), using 2-methoxybenzoic acid in place of 2,3-dimethoxybenzoic acid.

### Example 5

### Synthesis of N-benzyl-6-hydroxypyridine-2-carboxamide (compound 5)

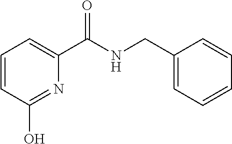

The title compound is prepared by the same method as for compound 2 (example 2), using 6-methoxypyridine-2-carboxylic acid in place of 2,3-dimethoxybenzoic acid.

### Example 6

### Synthesis of N-benzyl-2-hydroxypyridine-3-carboxamide (compound 6)

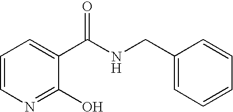

The title compound is prepared by the same method as for compound 2 (example 2), using 2-methoxypyridine-3-carboxylic acid in place of 2,3-dimethoxybenzoic acid.

### Example 7

### Synthesis of methyl 4-(2-methoxybenzylcarbamoyl)-2,3-dihydroxybenzoate (compound 7)

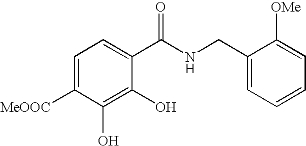

The title compound is prepared by the same method as for compound 1 (example 1), using (2-methoxyphenyl)methanamine in place of benzyl amine; ¹H NMR (400 MHz, CDCl₃) δ 11.8 (s, 1H), 10.9 (s, 1H), 7.34 (d, 1H), 7.31 (t, 1H), 7.29 (d, 1H), 7.12 (br, 1H), 6.96 (t, 1H), 6.92 (d, 2H), 4.64 (d, 2H), 3.97 (s, 3H), 3.91 (s, 3H); ESMS m/z 332 [M+H]⁺.

### Example 8

### Synthesis of methyl 4-(3-methoxybenzylcarbamoyl)-2,3-dihydroxybenzoate (compound 8)

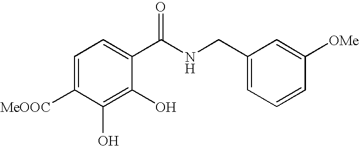

The title compound is prepared by the same method as for compound 1 (example 1), using (3-methoxyphenyl)methanamine in place of benzyl amine; ¹H NMR (400 MHz, CDCl₃) δ 11.2 (s, 1H), 10.9 (s, 1H), 7.32 (d, 1H), 7.27 (t, 1H), 7.03 (d, 1H), 6.94 (d, 1H), 6.95 (br, 1H), 6.89 (s, 1H), 6.86 (dd, 1H), 4.63 (d, 2H), 3.98 (s, 3H), 3.81 (s, 3H); ESMS m/z 332 [M+H]⁺.

### Example 9

### Synthesis of methyl 4-(4-methoxybenzylcarbamoyl)-2,3-dihydroxybenzoate (compound 9)

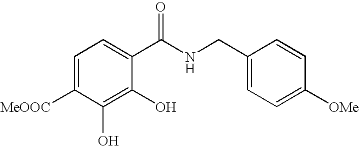

The title compound is prepared by the same method as for compound 1 (example 1), using (4-methoxyphenyl)methanamine in place of benzyl amine; ¹H NMR (400 MHz, CDCl₃) δ 11.3 (s, 1H), 10.9 (s, 1H), 7.31 (d, 1H), 7.29 (d, 2H), 6.99 (d, 1H), 6.90 (d, 2H), 6.85 (br, 1H), 4.59 (d, 2H), 3.97 (s, 3H), 3.81 (s, 3H); ESMS m/z 332 [M+H]⁺.

### Example 10

### Synthesis of methyl 4-(2-nitrobenzylcarbamoyl)-2,3-dihydroxybenzoate (compound 10)

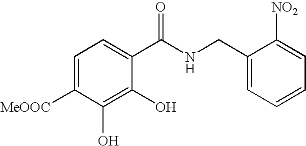

The title compound is prepared by the same method as for compound 1 (example 1), using (2-nitrophenyl)methanamine in place of benzyl amine; ¹H NMR (400 MHz, CDCl₃) δ 11.0 (s, 1H), 10.9 (s, 1H), 8.11 (dd, 1H), 7.76 (dd, 1H), 7.71 (br, 1H), 7.66 (dt, 1H), 7.51 (dt, 1H), 7.33 (d, 1H), 7.06 (d, 1H), 4.89 (d, 2H), 3.97 (s, 3H); ESMS m/z 347 [M+H]⁺.

### Example 11

### Synthesis of methyl 4-(3-nitrobenzylcarbamoyl)-2,3-dihydroxybenzoate (compound 11)

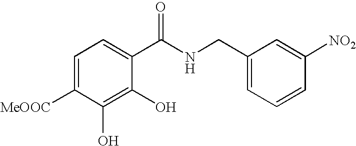

The title compound is prepared by the same method as for compound 1 (example 1), using (3-nitrophenyl)methanamine in place of benzyl amine; ¹H NMR (400 MHz, CDCl₃) δ 11.0 (s, 1H), 9.99 (s, 1H), 8.22 (s, 1H), 8.17 (d, 2H), 7.73 (d, 1H), 7.55 (t, 1H), 7.46 (br, 1H), 7.38 (d, 1H), 7.22 (d, 1H), 4.77 (d, 2H), 3.99 (s, 3H); ESMS m/z 347 [M+H]⁺.

### Example 12

### Synthesis of methyl 4-(4-nitrobenzylcarbamoyl)-2,3-dihydroxybenzoate (compound 12)

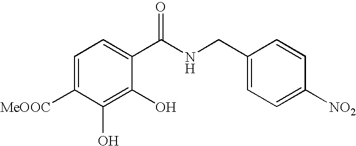

The title compound is prepared by the same method as for compound 1 (example 1), using (4-nitrophenyl)methanamine in place of benzyl amine; ¹H NMR (400 MHz, CDCl₃) δ 11.0 (s, 1H), 9.87 (s, 1H), 8.22 (d, 2H), 7.53 (d, 2H), 7.47 (br, 1H), 7.38 (d, 1H), 7.23 (d, 1H), 4.78 (d, 2H), 3.99 (s, 3H); ESMS m/z 347 [M+H]⁺.

### Example 13

### Synthesis of methyl 4-(2-(trifluoromethyl)benzylcarbamoyl)-2,3-dihydroxybenzoate (compound 13)

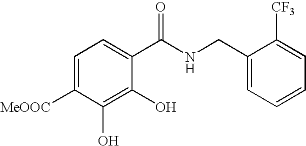

The title compound is prepared by the same method as for compound 1 (example 1), using (2-(trifluoromethyl)phenyl)methanamine in place of benzyl amine; ¹H NMR (400 MHz, CDCl₃) δ 11.0 (s, 1H), 10.7 (s, 1H), 7.69 (d, 1H), 7.64 (d, 1H), 7.56 (t, 1H), 7.43 (t, 1H), 7.33 (d, 1H), 7.17 (br, 1H), 7.07 (d, 1H), 4.84 (d, 2H), 3.98 (s, 3H); ESMS m/z 370 [M+H]⁺.

### Example 14

### Synthesis of methyl 4-(3-(trifluoromethyl)benzylcarbamoyl)-2,3-dihydroxybenzoate (compound 14)

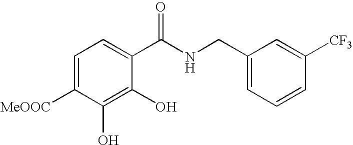

The title compound is prepared by the same method as for compound 1 (example 1), using (3-(trifluoromethyl)phenyl)methanamine in place of benzyl amine; ¹H NMR (400 MHz, CDCl₃) δ 11.0 (s, 1H), 10.4 (s, 1H), 7.60 (s, 1H), 7.57 (d, 2H), 7.49 (t, 1H), 7.36 (d, 1H), 7.24 (br, 1H), 7.15 (d, 1H), 4.73 (d, 2H), 3.98 (s, 3H); ESMS m/z 370 [M+H]⁺.

### Example 15

### Synthesis of methyl 4-(4-(trifluoromethyl)benzylcarbamoyl)-2,3-dihydroxybenzoate (compound 15)

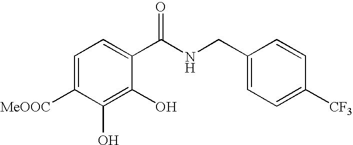

The title compound is prepared by the same method as for compound 1 (example 1), using (4-(trifluoromethyl)phenyl)methanamine in place of benzyl amine; ¹H NMR (400 MHz, CDCl₃) δ 11.0 (s, 1H), 10.4 (s, 1H), 7.62 (d, 2H), 7.48 (d, 2H), 7.36 (d, 1H), 7.24 (br, 1H), 7.15 (d, 1H), 4.73 (d, 2H), 3.98 (s, 3H); ESMS m/z 370 [M+H]⁺.

### Example 16

### Synthesis of methyl 4-((pyridin-2-yl)methylcarbamoyl)-2,3-dihydroxybenzoate (compound 16)

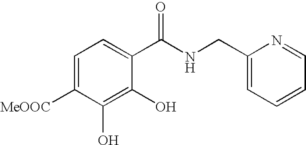

The title compound is prepared by the same method as for compound 1 (example 1), using (pyridin-2-yl)methanamine in place of benzyl amine; ¹H NMR (400 MHz, DMSO-d6) δ 12.4 (s, 1H), 10.4 (s, 1H), 9.59 (t, 1H), 8.60 (d, 1H), 7.96 (t, 1H), 7.52 (d, 1H), 7.74 (t, 1H), 7.44 (d, 1H), 7.28 (d, 1H), 4.68 (d, 2H), 3.91 (s, 3H); ESMS m/z 303 [M+H]⁺.

### Example 17

### Synthesis of methyl 4-((pyridin-3-yl)methylcarbamoyl)-2,3-dihydroxybenzoate (compound 17)

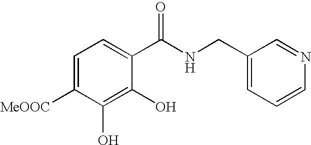

The title compound is prepared by the same method as for compound 1 (example 1), using (pyridin-3-yl)methanamine in place of benzyl amine; ¹H NMR (400 MHz, DMSO-d6) δ 12.4 (s, 1H), 10.4 (s, 1H), 9.55 (t, 1H), 8.71 (s, 1H), 8.62 (d, 1H), 8.06 (d, 1H), 7.64 (dd, 1H), 7.39 (d, 1H), 7.26 (d, 1H), 4.60 (d, 2H), 3.90 (s, 3H); ESMS m/z 303 [M+H]⁺.

### Example 18

### Synthesis of methyl 4-((pyridin-4-yl)methylcarbamoyl)-2,3-dihydroxybenzoate (compound 18)

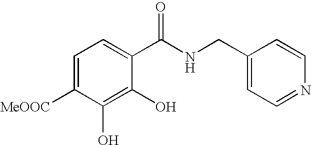

The title compound is prepared by the same method as for compound 1 (example 1), using (pyridin-4-yl)methanamine in place of benzyl amine; ¹H NMR (400 MHz, DMSO-d6) δ 12.3 (s, 1H), 10.5 (s, 1H), 9.61 (t, 1H), 8.71 (d, 2H), 7.69 (d, 2H), 7.42 (d, 1H), 7.29 (d, 1H), 4.68 (d, 2H), 3.91 (s, 3H); ESMS m/z 303 [M+H]⁺.

### Example 19

### Synthesis of methyl 4-(N-benzyl-N-methylcarbamoyl)-2,3-dihydroxybenzoate (compound 19)

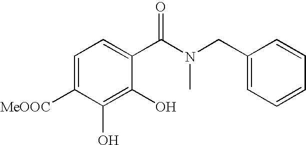

The title compound is prepared by the same method as for compound 1 (example 1); ¹H NMR (400 MHz, DMSO-d6) δ 10.7 (s, 1H), 9.64, 9.61 (2s, 1H), 7.1-7.4 (m, 6H), 6.76 (d, 1H), 4.68, 4.33 (2s, 2H), 3.91, 3.89 (2s, 3H), 2.84, 2.72 (2s, 3H); ESMS m/z 316 [M+H]⁺.

### Example 20

### Synthesis of methyl 4-(phenylcarbamoyl)-2,3-dihydroxybenzoate (compound 20)

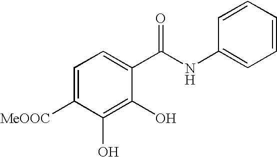

The title compound is prepared by the same method as for compound 1 (example 1); ¹H NMR (400 MHz, DMSO-d6) δ 11.6 (s, 1H), 10.5 (s, 2H), 7.70 (d, 2H), 7.43 (d, 1H), 7.38 (t, 2H), 7.31 (d, 1H), 7.15 (t, 1H), 3.91 (s, 3H); ESMS m/z 288 [M+H]⁺.

### Example 21

### Synthesis of 3-benzyl-8-hydroxy-2,4-dioxo-3,4-dihydro-2H-benzo[e][1,3]oxazine-7-carboxylic acid methyl ester (compound 21)

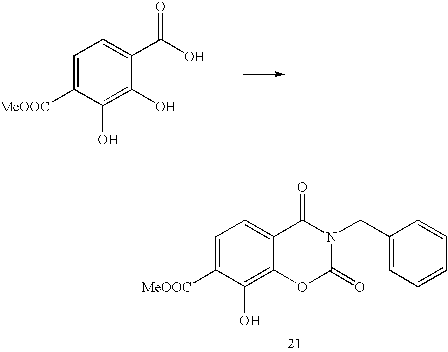

Ethyl chloroformate (0.35 ml, 3.5 mmol) is added dropwise to a solution of 2,3-dihydroxy-terephthalic acid monomethyl ester (prepared according to example 1; 212 mg, 1.0 mmol) and trietylamine (0.7 ml, 5.0 mmol) in dichloromethane (10 ml), at −10° C. The mixture is allowed to warm to room temperature for 3 hours, cooled to 0° C., and benzyl amine (0.44 ml, 4.0 mmol) is added. The mixture is stirred at room temperature overnight followed by removal of solvent under vacuum. The resulting residue is partitioned into ethyl acetate and water. The organic layer is separated and the aqueous layer is exracted with ethyl acetate. The combined organic layers are washed with brine, dried over Na₂SO₄, and evaporated to give the crude product which is purified on silica chromatography to afford the title compound (305 mg, 93%); ¹H NMR (400 MHz, CDCl₃) δ 11.2 (s, 1H), 7.78 (d, 1H), 7.54 (m, 3H), 7.32 (m, 3H), 5.21 (s, 2H), 4.02 (s, 3H); ¹³C NMR (400 MHz, CDCl₃) δ 169.5, 160.0, 150.0, 147.3, 142.0, 135.3, 129.4 (2C), 128.6 (2C), 128.3, 125.2, 118.6, 117.4, 116.2, 53.2, 46.0. MS m/z 328 [M+H]⁺.

Note: The structure of compound 21 is established based on HMBC and ROESY studies: a proton peak at chemical shift 11.2 ppm shows an HMBC correlation peak to carbons 12, 15 and 13 which is not possible in the alternate structure given below. The proton at carbon 7 has a correlation peak to 4 different carbons in the HMBC which is also not possible in the alternate structure. Finally, there is no visible cross-peak from proton at 11.2 ppm to the proton at carbon 7 in the ROESY which is expected.

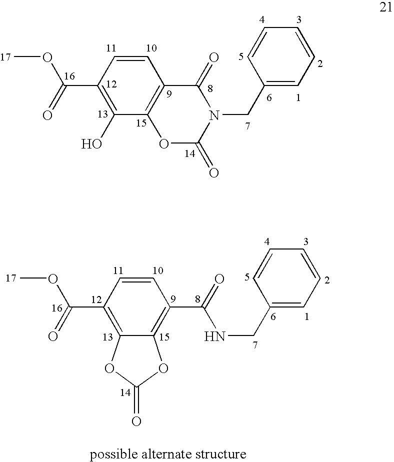

### Example 22

### Investigation of Anti-HIV Activity

HIV therapeutic agents inhibit propagation of HIV in cells, and as such cell-based assays of HIV antiviral activity have been developed. For example, Pauwels et al, Nature (1990) 343: 470-4 describe incubating HIV infected cells with test compounds and subsequently determining cell viability via colorimetric methods, to give an EC₅₀ for the inhibition of HIV-1 replication.

Once anti-HIV activity is observed, mechanism of action assays may be performed to determine how the therapeutic agent is inhibiting cell propagation. Compounds 1-21, as described herein, are screened in 3 different assays to assess their anti-HIV activity:

Screen 1: A high throughput cell-based HIV luciferase reporter infection assay (see He et al, Bioorg. Med. Chem. Lett. (2006) 16) that identifies inhibitors of early HIV infection events. The results are shown in Table 2 below, expressed as EC₅₀ (μM), the molar concentration that produces 50% of the maximal possible response.

Screen 2: An HIV-1 integrase strand transfer assay, (see Wang et al, J. Biomol. Screen. (2005) 10: 456), that identifies anti-HIV activity due to inhibition of HIV integrase. The results are shown in Table 2 below, expressed as IC₅₀ (μM), the molar concentration that produces 50% of the maximal possible inhibitory response.

Screen 3: A cytotoxicity assay, to determine inhibitory activity against HE 293T cells. The results are shown in Table 2.

TABLE 2
Anti-HIV and cytotoxicity evaluation of compounds 1-21

| | HIV Activity | Integrase Activity | Cytotoxicity |
|---|---|---|---|
| Compound | EC₅₀ | IC₅₀ | CC₅₀ |
| NVP^{a} | ++ | ++++ | ++++ |
| DKA^{b} | ++ | ++ | ++++ |
| 1 | ++ | ++ | ++++ |
| 2 | ++++ | — | ++++ |
| 3 | ++++ | — | ++++ |
| 4 | ++++ | — | ++++ |
| 5 | ++++ | — | ++++ |
| 6 | ++++ | — | ++++ |
| 7 | + | + | +++ |
| 8 | ++ | + | ++++ |
| 9 | +++ | + | ++++ |
| 10 | ++ | + | +++ |
| 11 | + | + | +++ |
| 12 | ++ | ++ | +++ |
| 13 | ++ | ++ | +++ |
| 14 | +++ | ++ | +++ |
| 15 | +++ | +++ | +++ |
| 16 | ++ | ++ | ++++ |
| 17 | ++ | ++ | ++++ |
| 18 | ++ | ++ | ++++ |
| 19 | ++++ | ++++ | ++++ |
| 20 | + | + | +++ |
| 21 | ++ | ++ | ++++ |

+ values less than 50 nM;
++ values from about 50 to about 500 nM;
+++ values from about 500 to about 2000 nM;
++++ value greater than 2000 nM;
— not determined.
^{a}Nevirapine (NVP), a non-nucleoside reverse transcriptase inhibitor
(NNRTI)
^{b}Diketoacid (DKA), an HIV integrase inhibitor (see Young et. al.,
WO 9962520) having the structure of

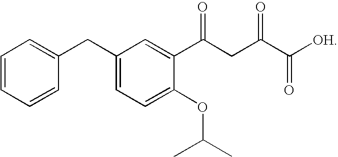

### Example 23

### Activity Against NNRTI Resistant Mutants

Select compounds are assayed against key NNRTI resistant mutants. The results are shown in Table 3, below.

TABLE 3
Inhibition of NNRTI resistant mutants (EC₅₀)

| Cpd | | WT | Y188L | Y181C | K103N | L100I |
|---|---|---|---|---|---|---|
| NVP | 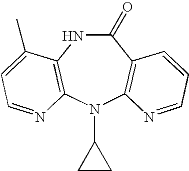 | ++ | ++++ | ++++ | ++++ | ++ |
| DKA | 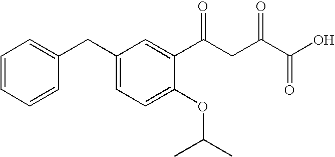 | ++ | ++ | ++ | + | ++ |
| 7 | 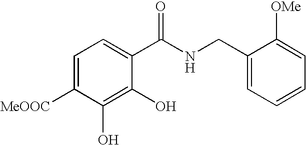 | + | ++ | ++ | + | + |
| 8 | 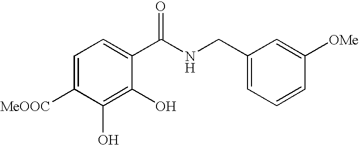 | ++ | ++ | ++ | ++ | ++ |
| 10 | 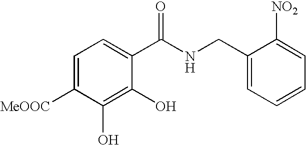 | ++ | ++ | ++ | + | + |
| 11 | 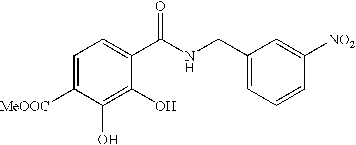 | + | + | ++ | + | + |
| 16 | 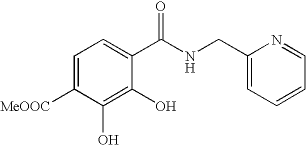 | ++ | ++ | ++ | ++ | ++ |
| 20 | 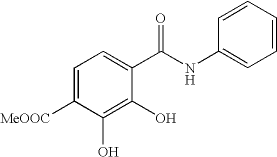 | + | + | + | + | + |
| 21 | 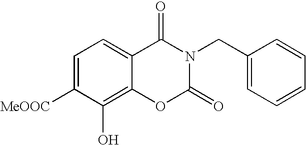 | ++ | ++ | ++ | ++ | ++ |

+ values less than 50 nM;
++ values from about 50 to about 500 nM;
+++ values from about 500 to about 2000 nM;
++++ value greater than 2000 nM;
— not determined.

### Example 24

### Molecular Modeling Studies: Docking Compound 1 With HIV-1 Integrase

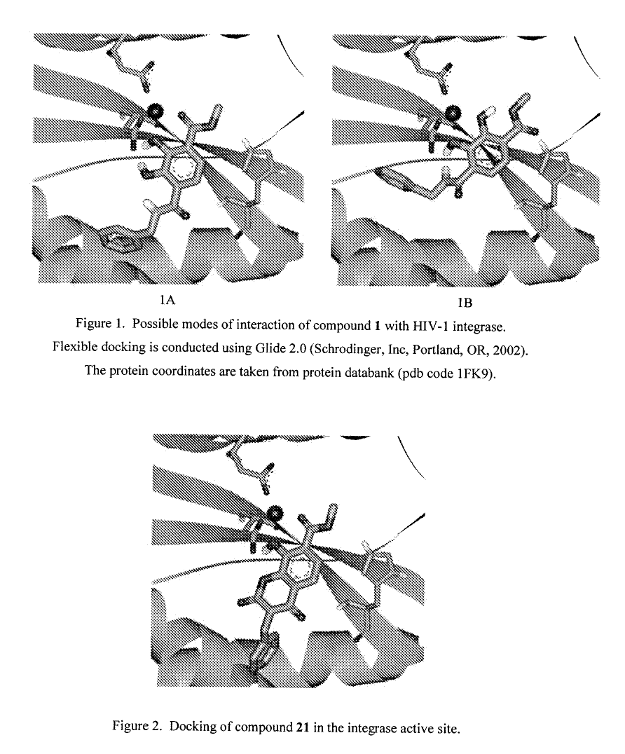

Molecular modeling studies are carried out in an attempt to better understand the interactions between the inhibitors described herein and the HIV integrase protein. Flexible docking studies are conducted using Glide 2.0 (Schrodinger, Inc, Portland, Oreg., 2002), using protein coordinates from the protein databank (pdb code 1FK9). The studies suggest two major possible binding modes for compound 1 with the integrase (FIG. 1). In the first model (FIG. 1A), the molecule coordinates with a metal ion via the ester and neighboring hydroxyl groups. The amide nitrogen makes an internal hydrogen bond with its neighboring hydroxyl group, which in turn is engaged in hydrogen bonding with D64.

In the second model (FIG. 1B), both hydroxyl groups coordinate the metal ion, and residue D64 is hydrogen bonded with the amide nitrogen and neighboring hydroxyl group. Both models imply explicit coordination between compound 1 and a metal ion, and the importance of the amide nitrogen either by rigidifying the compound via internal hydrogen bonds (as in model A) or due to hydrogen bonding with residue D64 (model B).

### Example 25

### Molecular Modeling Studies: Docking Compound 21 With HIV-1 Integrase

To further investigate the interaction modes, a rigidified compound 21, is prepared, in which the amide and its neighboring hydroxyl group are connected via a carbonyl group, to form a six-membered ring. Biological testing indicates that compound 21 maintains activity in both the cellular and enzymatic assays, (see Table 2, example 22 above). Though not wishing to be bound by any particular theory, the fact that compound 21 maintains activity despite lacking the amide hydrogen, suggests that model 1A is the more probable, since the role of the nitrogen in that model is to rigidify the structure via internal hydrogen bonding with the hydroxyl group. The same effect is achieved in compound 21. In contrast, model 2B suggests that the amide is engaged in hydrogen bonding with D64, and thus its removal would result in a decrease in activity.

Further, the energies of the protein-ligand complexes are calculated using Prime (Schrodinger, Inc.). The protein-ligand complex in 1A is calculated to be approximately 7 kcal/mol lower than for 1B, which further supports 1A being the more likely model for the interactions between the compounds of Formula (I), (II) or (III) and the HIV integrase enzyme.

It is understood that the examples and embodiments described herein are for illustrative purposes only and that various modifications or changes in light thereof can be suggested to persons skilled in the art and are to be included within the spirit and purview of this application and scope of the appended claims. All publications, patents, and patent applications cited herein are hereby incorporated by reference for all purposes.

1. A compound having the structure of Formula (I): 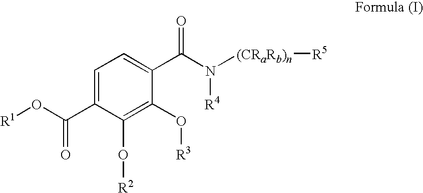
wherein
R¹ is H, alkyl or substituted alkyl;
R² is H, alkyl, substituted alkyl, —C(O)-alkyl or —C(O)-substituted alkyl;
R³ is H, alkyl, substituted alkyl, —C(O)-alkyl or —C(O)-substituted alkyl;
R⁴ is H, alkyl or substituted alkyl;
or —O—R³—R⁴—N— together form an optionally substituted, 6 or 7 membered ring;
Rₐ is H, halogen, C₁-C₆ alkyl or C₁-C₆ substituted alkyl;
R_{b} is H, halogen, C₁-C₆ alkyl or C₁-C₆ substituted alkyl;
R⁵ is optionally substituted C₃-C₅ cycloalkyl, optionally substituted lower heterocycloalkyl, optionally substituted aryl or optionally substituted heteroaryl;
where each substituent is independently selected from the group consisting of halogen, —CN, —NO₂, —N₃, ═O, ═S, ═NH, —SO₂, nitroalkyl, amino, dialkylamino, diarylamino, diarylalkylamino, cyanato, isocyanato, thiocyanato, isothiocyanato, guanidinyl, O-carbamyl, N-carbamyl, thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, -L¹-H, -L¹-alkyl, -L¹-substituted alkyl, -L¹-heteroalkyl, -L¹-haloalkyl, -L¹-perhaloalkyl, -L¹-alkenyl, -L¹-substituted alkenyl, -L¹-heteroalkenyl, -L¹-haloalkenyl, -L¹-perhaloalkenyl, -L¹-alkynyl, -L¹-substituted alkynyl, -L¹-heteroalkynyl, -L¹-haloalkynyl, -L¹-perhaloalkynyl, -L¹-cycloalkyl, -L¹-substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-unsubstituted aryl, -L¹-heteroaryl and -L¹-substituted heteroaryl;
where -L¹- is a bond, -alkylene-, -heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—, —C(S)NH—, —NHC(S)—, —S(O)—, —S(O)₂— or —S(O)NH—;
n is 0, 1 or 2;
and a pharmaceutically acceptable salt, pharmaceutically acceptable N-oxide, pharmaceutically active metabolite, pharmaceutically acceptable prodrug, pharmaceutically acceptable solvate thereof.

2. The compound of claim 1, wherein R¹ is alkyl.

3. The compound of claim 1, wherein R² is H.

4. The compound of claim 1, wherein R³ is H.

5. The compound of claim 1, wherein R² and R³ are H.

6. The compound of claim 1, wherein R⁴ is H.

7. The compound of claim 1, wherein n is 0.

8. The compound of claim 1, wherein n is 1.

9. The compound of claim 1, wherein R⁵ is optionally substituted aryl or optionally substituted heteroaryl.

10. The compound of claim 1, wherein R⁵ is substituted aryl or optionally substituted heteroaryl.

11. The compound of claim 1, wherein R5 is substituted phenyl or optionally substituted pyridyl.

12. The compound of claim 1, wherein R⁵ is an unsubstituted phenyl or an unsubstituted pyridyl.

13. The compound of claim 1, wherein R⁵ is substituted with at least one group selected from C₁-C₆ alkoxy, C₁-C₆ alkyl, C₁-C₆ haloalkyl, OH, NO₂, or NH₂.

14. The compound of claim 1, wherein R⁵ is selected from the group consisting of:

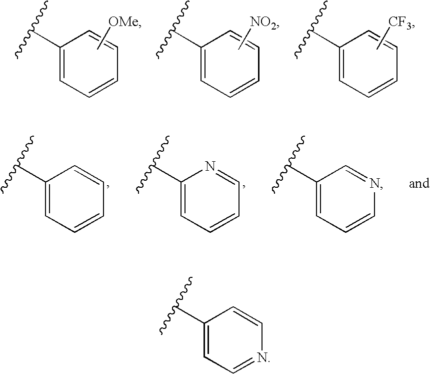

15. The compound of claim 1, wherein
R¹ is alkyl;
R²═R³═R⁴═H;
R⁵ is substituted phenyl or substituted pyridyl; and
n is 0 or 1.

16. The compound of claim 1, wherein
R¹ is alkyl;
R²═R³═R⁴═H;
R⁵ is unsubstituted phenyl or unsubstituted pyridyl; and
n is 0 or 1.

17. The compound of claim 1, wherein —O—R³—R⁴—N— together form an optionally substituted, 6 or 7 membered ring.

18-61. (canceled)

62. A pharmaceutical composition comprising at least one compound having the structure of Formula (I) or the respective pharmaceutically acceptable salts, pharmaceutically active metabolites, pharmaceutically acceptable prodrugs or pharmaceutically acceptable solvates, in admixture with one or more excipients; where the structure of Formula (I) is 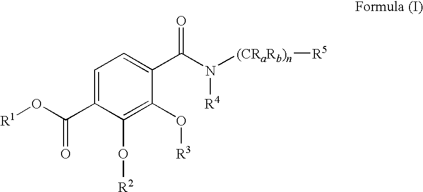
wherein
R¹ is H, alkyl or substituted alkyl,
R² is H, alkyl substituted alkyl —C(O)-alkyl or —C(O)-substituted alkyl;
R³ is H, alkyl, substituted alkyl, —C(O)-alkyl or —C(O)-substituted alkyl,
R⁴ is H, alkyl or substituted alkyl;
or —O—R³—R⁴—N— together form an optionally substituted, 6 or 7 membered ring;
Rₐ is H, halogen, C₁-C₆ alkyl or C₁-C₆ substituted alkyl;
R_{b} is H, halogen, C₁-C₆ alkyl or C₁-C₆ substituted alkyl,
R⁵ is optionally substituted C₃-C₅ cycloalkyl, optionally substituted lower heterocycloalkyl, optionally substituted aryl or optionally substituted heteroaryl;
where each substituent is independently selected from the group consisting of halogen, —CN, —NO₂, —N₃, ═O, ═S, ═NH, —SO₂, nitroalkyl, amino, dialkylamino, diarylamino, diarylalkylamino, cyanato, isocyanato, thiocyanato, isothiocyanato, guanidinyl, O-carbamyl, N-carbamyl thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, -L¹-H, -L¹-alkyl, -L¹-substituted alkyl, -L¹-heteroalkyl, -L¹-haloalkyl, -L¹-perhaloalkyl,
-L¹-alkenyl, -L¹-substituted alkenyl, -L¹-heteroalkenyl, -L¹-haloalkenyl, -L¹-perhaloalkenyl, -L¹-alkynyl,
-L¹-substituted alkynyl, -L¹-heteroalkynyl, -L¹-haloalkynyl, -L¹-perhaloalkynyl, -L¹-cycloalkyl, -L¹-substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-unsubstituted aryl, -L¹-heteroaryl and -L¹-substituted heteroaryl,
where -L¹- is a bond, -alkylene-, -heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—, —C(S)NH—, —NHC(S)—, —S(O)—, —S(O)₂— or —S(O)NH—, and
n is 0, 1 or 2.

63. (canceled)

64. (canceled)

65. A method of preventing, inhibiting or ameliorating the pathology and/or symptomology of infection with an immunodeficiency virus in an animal, comprising administering to said animal a therapeutically effective amount of at least one compound of Formula (I) or the respective pharmaceutically acceptable salts, pharmaceutically active metabolites, pharmaceutically acceptable prodrugs or pharmaceutically acceptable solvates; where the structure of Formula (I) is 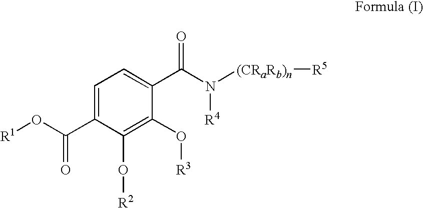
wherein
R¹ is H, alkyl or substituted alkyl,
R² is H, alkyl substituted alkyl —C(O)-alkyl or —C(O)-substituted alkyl;
R³ is H, alkyl, substituted alkyl, —C(O)-alkyl or —C(O)-substituted alkyl,
R⁴ is H, alkyl or substituted alkyl;
or —O—R³—R⁴—N— together form an optionally substituted, 6 or 7 membered ring;
Rₐ is H, halogen, C₁-C₆ alkyl or C₁-C₆ substituted alkyl;
R_{b} is H, halogen, C₁-C₆ alkyl or C₁-C₆ substituted alkyl;
R⁵ is optionally substituted C₃-C₅ cycloalkyl, optionally substituted lower heterocycloalkyl, optionally substituted aryl or optionally substituted heteroaryl;
where each substituent is independently selected from the group consisting of halogen, —CN, —NO₂, —N₃, ═O , ═S, ═NH, —SO₂, nitroalkyl, amino, dialkylamino, diarylamino, diarylalkylamino, cyanato, isocyanato, thiocyanato, isothiocyanato, guanidinyl, O-carbamyl, N-carbamyl thiocarbamyl, uryl, isouryl, thiouryl, isothiouryl, mercapto, sulfanyl, sulfinyl, sulfonyl, sulfonamidyl, phosphonyl, phosphatidyl, phosphoramidyl, -L¹-H, -L¹-alkyl, -L¹-substituted alkyl, -L¹-heteroalkyl, -L¹-haloalkyl, -L¹-perhaloalkyl,
-L¹-alkenyl, -L¹-substituted alkenyl, -L¹-heteroalkenyl, -L¹-haloalkenyl, -L¹-perhaloalkenyl, -L¹-alkynyl,
-L¹-substituted alkynyl, -L¹-heteroalkynyl, -L¹-haloalkynyl, -L¹-perhaloalkynyl, -L¹-cycloalkyl, -L¹-substituted cycloalkyl, -L¹-heterocycloalkyl, -L¹-substituted heterocycloalkyl, -L¹-cycloalkenyl, -L¹-substituted cycloalkenyl, -L¹-heterocycloalkenyl, -L¹-substituted heterocycloalkenyl, -L¹-cycloalkynyl, -L¹-substituted cycloalkynyl, -L¹-heterocycloalkynyl, -L¹-substituted heterocycloalkynyl, -L¹-unsubstituted aryl, -L¹-heteroaryl and -L¹-substituted heteroaryl;
where -L¹- is a bond, -alkylene-, -heteroalkylene-, -alkenylene-, -alkynylene-, -arylene-, -heteroarylene-, —O—, —S—, —NH—, —C(O)—, —C(S)—, OC(O)—, —C(O)O—, SC(O)—, —C(S)O—, —C(O)NH—, —NHC(O)—, —C(S)NH—, —NHC(S)—, —S(O)—, —S(O)₂— or —S(O)NH—, and
n is 0, 1 or 2.

66-101. (canceled)

102. The compound of claim 1 selected from 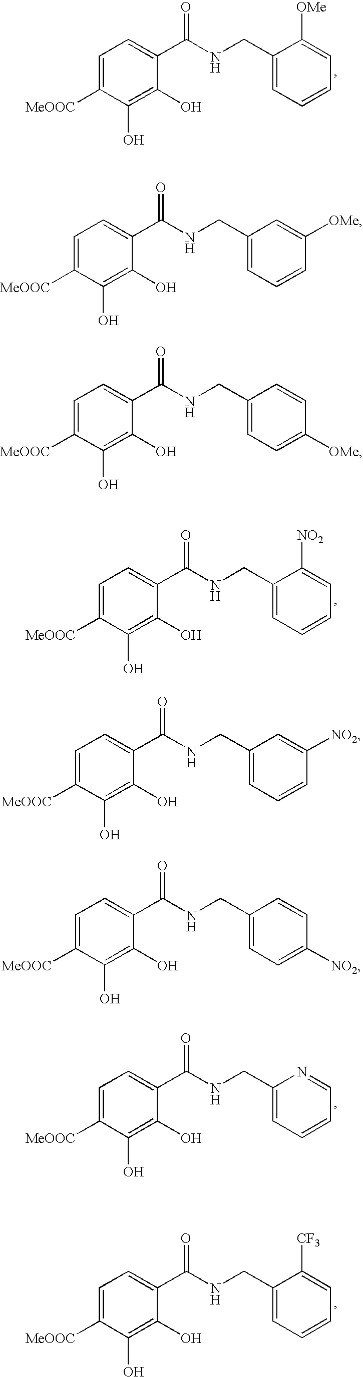 [chemical structure]
